(19) United States
(12) Patent Application Publication
(10) Pub. No.: US 2022/0072110 A1
(43) Pub. Date: Mar. 10, 2022

(54) PLASMINOGEN FOR TREATING AND PREVENTING MICROTHROMBOSIS

(71) Applicant: PREVIPHARMA CONSULTING GMBH, Mannheim (DE)

(72) Inventors: Stephan T. KIESSIG, Wiesloch (DE); Ricarda WELZ, Mauer (DE); Hanne Rieke GERDING, Mannheim (DE); Marc MAZUR, Weinheim (DE); Hans Joachim ANDERS, Muenchen (DE); Chongxu SHI, Muenchen (DE); Christoph SCHIMMELPFENNIG, Leipzig (DE); Satish Kumar DEVARAPU, Muenchen (DE)

(21) Appl. No.: 17/422,962

(22) PCT Filed: Jan. 24, 2020
(86) PCT No.: PCT/EP2020/051733

§ 371 Date: Jul. 14, 2021

(30) Foreign Application Priority Data
Jan. 24, 2019 (EP) 19153572.3

(51) Int. Cl. A61K 38/48

(52) U.S. Cl. CPC A61K 38/484; C12Y 304/21007

(57) ABSTRACT

The present invention relates to plasminogen for use in a method for preventing or treating a thrombotic event in a patient, wherein the patient is at risk of developing or is suffering from microthrombi.

The present invention relates to plasminogen (PLG) for use in a method for preventing or treating a thrombotic event in a patient, wherein the patient is at risk of developing or is suffering from microthrombi. The present invention also refers to plasminogen (PLG) for use in a method for the lysis of microthrombi and in the prophylaxis of microthrombi in a patient having an inborn or acquired plasminogen (PLG) deficiency.

Thrombotic events, in particular microthrombi, can cause serious health problems. For instance, coronary infarcts, stroke and pulmonary embolisms are some of the main causes of death in the developed world. Therefore, there is a strong need to treat and prevent thrombotic events. In particular microthrombi, i.e., thrombotic events of small size, in particular occurring in the capillary system, are not easily treatable in a patient.

The formation of a microthrombotic event may have various reasons such as, for example, surgical interventions such as a vascular surgery (e.g., coronary bypass surgery), the presence of an endoprosthesis (i.e., insertion of foreign bodies/implants or transplants, e.g., blood vessel/vascular endoprosthesis), or a stenosis traumatic injury. A variety of further causes are known.

The treatment of thromboses is usually by administration of medicaments. In order to prevent the thrombus from enlarging, an anticoagulation inhibition is sought. Heparin preparations or factor Xa inhibitors are used. When the growth of the clot stops, the body can begin to clean up the damage. The body typically breaks down the clot and tries to get the veins free (re-vasculated) again. This typically takes several days, weeks or even months, the more regions of the venous system were affected, the longer. In the breakdown of the clot and the regeneration of the veins, substances are released, which increase the coagulability of the blood.

During this time, the risk of a renewed/repeated thrombotic event is particularly high. Therefore, further anticoagulant drugs are often avoided. 4-Hydroxycoumarins such as phenprocoumone, warfarin or ethylbiscoumacetat are used for about three to six months. The use of cumarins typically requires regular blood tests and special attention, because the drugs prevent thrombosis, but also increase the willingness to bleed. Above all, this risk of bleeding due to anticoagulant therapy is still an unsolved problem in everyday clinical practice. Despite of the antithrombotic therapy a residual amount of coagulation in the diseases mentioned above is still needed. First, after all surgeries, the wound healing requires this to stop the bleeding into the wounds. The clot itself is the base for the invasion of cells which are needed for the tissue repair (fibroblasts). They are using the fibrin network to clamp the wound, to build new collagen fibers, to re(build) the new tissue. The wound healing typically has four phases:

Phase I—Hemostasis Phase: is the process of the wound being closed by clotting. Hemostasis starts when blood leaks out of the body. The first step of hemostasis is when blood vessels constrict to restrict the blood flow. Next, platelets stick together in order to seal the break in the wall of the blood vessel. Finally, coagulation occurs and reinforces the platelet plug with threads of fibrin which are like a molecular binding agent. The hemostasis stage of wound healing happens very quickly. The platelets adhere to the sub-endothelium surface within seconds of the rupture of a blood vessel's epithelial wall. After that, the first fibrin strands begin to adhere in about sixty seconds. As the fibrin mesh begins, the blood is transformed from liquid to gel through pro-coagulants and the release of prothrombin. The formation of a thrombus or clot keeps the platelets and blood cells trapped in the wound area. The thrombus is generally important in the stages of wound healing but becomes a problem if it detaches from the vessel wall and goes through the circulatory system, possibly causing a stroke, pulmonary embolism or heart attack. It is known that faster fibrin clot degradation characterizes patients with central pulmonary embolism at a low risk of recurrent peripheral embolism (cf. Kupis et al, Scientific Reports, 2019, 9:72).

Phase II—Inflammatory Phase: Inflammation is the second stage of wound healing and begins right after the injury when the injured blood vessels leak transudate (made of water, salt, and protein) causing localized swelling. Inflammation both controls bleeding and prevents infection. The fluid engorgement allows healing and repair cells to move to the site of the wound.

During the inflammatory phase, damaged cells, pathogens, and bacteria are removed from the wound area. These white blood cells, growth factors, nutrients and enzymes create the swelling, heat, pain and redness commonly seen during this stage of wound healing. Inflammation is a natural part of the wound healing process and only problematic if prolonged or excessive.

Phase III—Proliferative Phase: The proliferative phase of wound healing is when the wound is rebuilt with new tissue made up of collagen and extracellular matrix. In the proliferative phase, the wound contracts as new tissues are built. In addition, a new network of blood vessels must be constructed so that the granulation tissue can be healthy and receive sufficient oxygen and nutrients. Myofibroblasts cause the wound to contract by gripping the wound edges and pulling them together using a mechanism similar to that of smooth muscle cells. In healthy stages of wound healing, granulation tissue is pink or red and uneven in texture. Moreover, healthy granulation tissue does not bleed easily. Dark granulation tissue can be a sign of infection, ischemia, or poor perfusion. In the final phase of the proliferative stage of wound healing, epithelial cells resurface the injury. It is important to remember that epithelialization happens faster when wounds are kept moist and hydrated. Generally, when occlusive or semi-occlusive dressings are applied within 48 hours after injury, they will maintain correct tissue humidity to optimize epithelialization.

Phase IV—Maturation Phase: Also called the remodeling stage of wound healing, the maturation phase is when collagen is remodeled from type III to type I and the wound fully closes. The cells that had been used to repair the wound but which are no longer needed are removed by apoptosis, or programmed cell death. When collagen is laid down during the proliferative phase, it is disorganized and the wound is thick. During the maturation phase, collagen is aligned along tension lines and water is reabsorbed so the collagen fibers can lie closer together and cross-link. Cross-linking of collagen reduces scar thickness and also makes the skin area of the wound stronger. Generally, remodeling begins about 21 days after an injury and can continue for a year or more. Even with cross-linking, healed wound areas continue to be weaker than uninjured skin, generally only having 80% of the tensile strength of unwounded skin.

The stages of wound healing are a complex and fragile process. Failure to progress in the stages of wound healing can lead to chronic wounds. Factors that lead up to chronic wounds are venous disease, infection, diabetes and metabolic deficiencies of the elderly. Despite of the anticoagulatory therapies, a residual coagulation activity is needed for the wound healing process as well as the fibrinolysis, which runs in parallel with the proliferative and the maturation phase.

Most anticoagulatory drugs known in the art have severe drawbacks and often cause significant health risks. Problems regarding coagulation can be caused by, e.g., an unknown liver insufficiency or by an overdosing of the anticoagulatory drugs. The fibrinolytic site is currently not in focus. An observation of both sides can help to improve the results of the treatments. An improved treatment is desired.

The currently used tPA (tissue plasminogen activator or uPA (Urokinase)) is applied as an indirect therapy requiring at least a normal plasminogen (PLG) level. tPA typically activates the plasminogen (PLG) to plasmin. This is used to dissolve an already formed thrombus which may cause consecutive additional tissue damages. Therapies can fail, if there is in insufficient amount of plasminogen (PLG) available. This was found in about one third of all patients under “lysis therapy” (Stubblefield W B, Alves N J, Rondina M T, Kline J A: Variable Resistance to Plasminogen (PLG) Activator Initiated Fibrinolysis for Intermediate-Risk Pulmonary Embolism. PLoS One. 2016 Feb. 11; 11(2):e0148747. doi: 10.1371/journal.pone.0148747. eCollection 2016; Cullis J O, Chisholm M, Ackery D M: Unresolved pulmonary embolism: the role of fibrinolysis. Nucl Med Commun. 1993 January; 14(1):4-7).

Mixtures of different types of plasminogen (PLG) also bear significant drawbacks when administered to the patient.

WO 2018/162754 teaches that Glu-plasminogen can be obtained from blood plasma and plasma fractions, in particular cryo-poor plasma, cryo-poor plasma reduced by one or two absorption steps (F IX/PCC or C1-Esterase Inhibitor), Paste or octanoic acid paste, and may serve as an effective pharmaceutical agent for treating a patient suffering from or being at risk of developing organ infarctions.

U.S. Pat. No. 5,597,800 teaches the treatment of brain edema by means of Lys-plasminogen. Also, general replacement therapies of administration of plasminogen (PLG) are described (cf. Shapiro et al., Blood, 2018, 131:1301-1310; Schott et al., The New England Journal of Medicine, 1998, 339:1679-1686).

WO 2017/077380 teaches a supplementation therapy of plasmin(ogen). This document does not teach the prevention or treatment of microthrombi having diameters of less than 1 mm. Furthermore, WO 2017/077380 does not seem to refer to Glu-plasminogen in the sense of the present invention, because plasmin(ogen) as used in the context of WO 2017/077380 bears significant proteolytic activity. In this context, WO 2017/077380 teaches that plasminogen as used therein degrades fibrin (cf. WO 2017/077380, page 25, lines 24 and 25, and page 30, line 24). In contrast, Glu-plasminogen in the sense of the present invention, in contrast to plasmin and Lys-plasminogen, has (essentially) no proteolytic activity.

EP-A 3395359 teaches a method for preventing and/or eliminating an arterial and venous thrombosis in a subject. Also this document does not teach the prevention or treatment of microthrombi having diameters of less than 1 mm. Furthermore, EP-A 3395359 does not refer to Glu-plasminogen. In contrast, plasminogen (PLG) as taught in EP-A 3395359 bears proteolytic activity, while Glu-plasminogen in the sense of the present invention has (essentially) no proteolytic activity. EP-A 3395359 is focused on proteins of 810 amino acids in lengths as plasminogen (PLG), thus a protein different from Glu-plasminogen.

It is, however, still particularly challenging to treat and prevent microthrombi, i.e., thrombotic events of small size of a diameter of less than 1 mm. The means for treating thrombi are mainly focused on the treatment of large-size thrombi after these occurred. There is, however, also a desire for prophylaxis of thrombotic events as well as for treating and preventing microthrombotic events in a patient.

Surprisingly, it was experimentally found that plasminogen (PLG), in particular Glu-plasminogen, can be very effectively used as a method for preventing or treating thrombotic events, in particular the formation of microthrombi, in a patient.

The present invention relates to plasminogen (PLG), in particular Glu-plasminogen, for use in a method for preventing or treating a thrombotic event in a patient. More specifically, the present invention relates to plasminogen (PLG) for use in a method for preventing or treating a thrombotic event in a patient, wherein the patient is at risk of developing or is suffering from microthrombi having diameters of less than 1 mm.

In other words, the present invention relates to a method for preventing or treating a thrombotic event in a patient, wherein said patient is administered with a sufficient amount of plasminogen (PLG), in particular Glu-plasminogen. More specifically, the present invention relates to a method for preventing or treating a thrombotic event in a patient, wherein said patient is administered with a sufficient amount of plasminogen (PLG) and wherein the patient is at risk of developing or is suffering from microthrombi having diameters of less than 1 mm.

An aspect of the present invention relates to plasminogen (PLG) for use in a method for preventing a microthrombotic event in a patient, wherein the patient is at risk of developing microthrombi having diameters of less than 1 mm.

Another aspect of the present invention relates to plasminogen (PLG) for use in a method for treating a microthrombotic event in a patient, wherein the patient is suffering from microthrombi having diameters of less than 1 mm.

Typically, the patient is suffering from or is at risk of developing a multitude of microthrombi. The patient is suffering from or is at risk of developing at least 10, at least 50, at least 100 microthrombi which in a thrombotic event.

According to the present invention, the thrombotic event is a microthrombus. As used herein, the term “microthrombus” (plural “microthrombi”) may be understood in the broadest sense as any thrombus having a maximal diameter in one of the spatial directions of less than 1 mm (millimeter), preferably less than 0.9 mm, less than 0.8 mm, less than 0.7 mm, less than 0.6 mm, less than 0.5 mm, less than 0.4 mm, less than 0.3 mm, less than 0.2 mm or less than 0.1 mm. It will be understood that the diameter of the remaining spatial directions may optionally independently from each other be larger or smaller. It will be generally understood that a maximal diameter is a theoretical cut length when theoretically cutting through the microthrombus where it has its broadest spatial extent in this direction. A microthrombus may also be a micro-coagulation disorder.

Accordingly, in a preferred embodiment, the present invention also relates to the plasminogen (PLG) for use in a method for preventing or treating microthrombosis in a patient.

Preferably, a microthrombus has maximal diameters in two of the three spatial directions of less than 1 mm, preferably less than 0.9 mm, less than 0.8 mm, less than 0.7 mm, less than 0.6 mm, less than 0.5 mm, less than 0.4 mm, less than 0.3 mm, less than 0.2 mm or less than 0.1 mm. It will be understood that the diameter of the remaining spatial direction may optionally be larger or smaller.

Preferably, a microthrombus has maximal diameters in all three spatial directions of less than 3 mm, preferably less than 2.5 mm, less than 2 mm, less than 1.5 mm, less than 1 mm or less than 0.5 mm, in particular when concomitantly having maximal diameters in two of the three spatial directions of less than 1 mm, preferably less than 0.9 mm, less than 0.8 mm, less than 0.7 mm, less than 0.6 mm, less than 0.5 mm, less than 0.4 mm, less than 0.3 mm, less than 0.2 mm or less than 0.1 mm.

Preferably, a microthrombus has maximal slice plane of less than 2 mm² (square millimeters), preferably of less than 1.5 mm², less than 1 mm², less than 0.9 mm², less than 0.8 mm², less than 0.7 mm², less than 0.6 mm², less than 0.5 mm², less than 0.4 mm², less than 0.3 mm², less than 0.2 mm² or less than 0.1 mm². It will be generally understood that a maximal slice plane is a theoretical cut surface area when theoretically cutting through the microthrombus where it has its broadest spatial extent.

In view of the above, in a preferred embodiment, the thrombotic event is a thrombus of a blood vessel having a diameter of less than 1 mm. In a preferred embodiment, the thrombotic event is a thrombus of a blood vessel having a diameter of less than 0.9 mm, less than 0.8 mm, less than 0.7 mm, less than 0.6 mm, less than 0.5 mm, less than 0.4 mm, less than 0.3 mm, less than 0.2 mm or less than 0.1 mm.

In a preferred embodiment, the thrombotic event is a thrombus of a capillary. In a preferred embodiment, the microthrombi are microthrombi of capillaries.

In general terms, the present invention relates to a plasminogen (PLG) and its derivatives or combinations (Glu-plasminogen, Lys-plasminogen and/or plasmin). The structure and function of the plasminogen (PLG)/plasmin system are generally known (cf. Aisina and Mukhametova, Russian Journal of Bioorganic Chemistry, 2014, 40:590-605).

In a particularly preferred embodiment, the plasminogen (PLG) as used in the context of the present invention is Glu-plasminogen. Therefore, throughout the present invention, plasminogen (PLG) may be specified as being Glu-plasminogen. In an alternative preferred embodiment, the plasminogen (PLG) is Lys-plasminogen. In an alternative preferred embodiment, the plasminogen (PLG) is a combination of Glu-plasminogen and Lys-plasminogen. Herein, Lys-plasminogen and Glu-plasminogen may be combined in any variation. In an alternative preferred embodiment, the plasminogen (PLG) is a combination of Glu-plasminogen and Lys-plasminogen and one or more other plasminogen (PLG) derivatives. Herein, Lys-plasminogen and Glu-plasminogen and the one or more other plasminogen (PLG) derivatives may be combined in any variation.

Glu-plasminogen is a plasma-derived proenzyme. It is known that Glu-plasminogen has (essentially) no proteolytic activity.

Preferably, the plasminogen composition (preferably containing Glu-plasminogen) administered to the patient has (essentially) no proteolytic activity.

Such (essential) absence in proteolytic activity may be understood in the broadest sense as generally understood by a person skilled in the art. Preferably, the enzymatic activity of Glu-plasminogen and/or the plasminogen composition (preferably containing Glu-plasminogen) administered to the patient is below 70 units (U, i.e., μmol/min) per 1.0 g/L of total protein content, or below 10, below 9, below 8, below 7, below 6, below 5, below 2, below 1, below 0.5, below 0.1, or below 0.01 U per 1.0 g/L of total protein content. In this context, proteolytic activity may be determined by any means. For instance, it may be the activity determined by an S-2288 (Chromogenix) proteolytic activity assay. Alternatively, it may also be determined as the degradation of fibrin to D-dimers. The specific enzyme activity of plasmin can be determined as degradation of fibrin to D-dimers. Preferably, proteolytic activity of Glu-plasminogen and/or the plasminogen composition (preferably containing Glu-plasminogen) administered to the patient is below the detection limit of the assay.

In a preferred embodiment, the plasminogen composition administered to the patient contains Glu-plasminogen in a purity of at least 90% (w/w), at least 95% (w/w), at least 97% (w/w), at least 98% (w/w), at least 99% (w/w), at least 99.5% (w/w), at least 99.7% (w/w), at least 99.8% (w/w), or at least 99.9% (w/w), based on the total protein content.

In a preferred embodiment, the plasminogen composition (preferably containing Glu-plasminogen) administered to the patient contains no or only a very low endotoxin content of below 1 EU/mL, below 0.5 EU/mL, below 0.1 EU/mL, below 0.05 EU/mL, or below 0.01 EU/mL (as determined in a Limulus Amebocyte Lysate (LAL) endosafe endochrome assay according to European Pharmacopeia (version 5.0) chapter 2.6.14).

In a preferred embodiment, the plasminogen composition (preferably containing Glu-plasminogen) administered to the patient contains no or only a very low immunoglobulin content of below 5 g/L, below 2 g/L, below 1 g/L, below 0.5 g/L, or below 0.1 g/L of immunoglobulins (determined in a nephelometric assay).

In a preferred embodiment, the plasminogen composition (preferably containing Glu-plasminogen) administered to the patient contains no or only a very low Lys-plasminogen content. In a preferred embodiment, the plasminogen composition (preferably containing Glu-plasminogen) administered to the patient contains no or only a very low albumin content.

In a preferred embodiment, the patient bears an acquired plasminogen (PLG) deficiency caused by increased plasminogen (PLG) consumption, decreased biosynthesis of Glu-plasminogen, or a combination of both. In a preferred embodiment, the patient bears an acquired Glu-plasminogen deficiency caused by increased Glu-plasminogen consumption, decreased biosynthesis of Glu-plasminogen, or a combination of both.

In a preferred embodiment, patient bears an acquired plasminogen (PLG) deficiency. In a preferred embodiment, patient bears an acquired plasminogen (PLG) deficiency caused by increased plasminogen (PLG) consumption (consumption hypofibrinolysis). Plasminogen (PLG) consumption may be due to transformation of the plasminogen (PLG) to plasmin. This may have any reason, for instance, by a long-term activation of the fibrinolytic system such as, e.g., due to clot formation and/or damage of one or more blood vessels' inside, the intima.

In a preferred embodiment, plasminogen (PLG) is Glu-plasminogen and the patient bears an acquired Glu-plasminogen deficiency. In a preferred embodiment, plasminogen (PLG) is Glu-plasminogen and the patient bears an acquired Glu-plasminogen deficiency caused by increased Glu-plasminogen consumption.

In a preferred embodiment, patient bears a decreased biosynthesis of plasminogen (PLG). In a preferred embodiment, patient bears a decreased biosynthesis of Glu-plasminogen. For instance, such decreased biosynthesis may be caused by decreased expression of messenger ribonucleic acid (mRNA) encoding for plasminogen (PLG), in particular Glu-plasminogen, erroneous splicing of such mRNA, reduced translation of the mRNA into the respective polypeptide, accelerated intracellular degradation of mRNA and/or the respective polypeptide, or a combination of two or more thereof.

As used herein, the terms “increased” and “decreased” refer to a comparison with the average of a healthy population. Preferably, “increased” may be a value that is at least 5%, at least 10%, at least 25%, at least 50% or at least 2 fold higher than the average in a healthy population. Preferably, “decreased” may be a value that is at least 5%, at least 10%, at least 25%, or at least 50% lower than the average in a healthy population.

As used herein, an acquired (Glu-)plasminogen deficiency may be understood in the broadest sense as a shortage of (Glu-)plasminogen as, for instance, acquired during patent's live. Thus, such (Glu-)plasminogen deficiency differs from an innate (Glu-)plasminogen deficiency. Preferably, an acquired (Glu-)plasminogen deficiency is caused by increased plasminogen (PLG) consumption. Such plasminogen (PLG) consumption may be caused by events in the patient's body as described herein.

In a preferred embodiment, plasminogen (PLG) as used in the context of the present invention has lower proteolytic activity than plasmin. In a preferred embodiment, plasminogen (PLG) as used in the context of the present invention has a low or no proteolytic activity. In a preferred embodiment, plasminogen (PLG) as used in the context of the present invention has (essentially) no proteolytic activity. A high proteolytic activity can be triggered by active enzymes like as tPA, plasmin or other active proteases which are preferably captured during the purification process. In other words, the plasminogen (PLG) as used in the context of the present invention does preferably (essentially) not contain a proteolytic ingredient. The plasminogen (PLG) as used in the context of the present invention does preferably (essentially) not contain tPA, plasm in or other active proteases.

The selective use of Glu-plasminogen is particularly beneficial in the context of the present invention. The blood coagulation is balanced by two inhibitors, antithrombin III and heparin cofactor II. Formed fibrin clots are only removed by activation of the fibrinolytic system. The activation of the fibrinolytic system is dependent on the plasmin activation. Human plasma contains plasminogen (PLG) in several forms of activation starting with Glu-plasminogen (native), Lys-plasminogen (slightly activated) and plasmin, in its activated form. The activation of the native Glu-plasminogen through uPA, tPA to Glu-plasmin in a healthy individual is a key mechanism (Stricker, R. B.; Wong, Activation of plasminogen (PLG) by tissue plasminogen (PLG) activator on normal and thrombasthenic platelets: effects on surface proteins and platelet aggregation. Blood 1986, S. 275-280).

Streptokinase or urokinase is used in therapeutic setting to achieve a thrombolysis in different thrombogenic events (Kunamneni, A.; Durvasula, R. Streptokinase-A Drug for Thrombolytic Therapy: A Patent Review. Recent advances in cardiovascular drug discovery 2014, S. 106-121; Takada, Akikazu; Takada, Yumiko, Activation pathway of Glu-plasminogen to Lys-plasmin by urokinase. Thrombosis research 1982, S. 671-677). The fibrinolysis is started due to the activation of plasminogen (PLG) leading to the cleavage from plasminogen (PLG) to plasmin [Wohl, R. C.; Kinetics of activation of human plasminogen (PLG) by different activator species at pH 7.4 and 37 degrees C., The Journal of Biological Chemistry 1980, S. 2005-2013]. Thereby three different activation mechanisms are known [Fredenburgh, J. C.; Nesheim, M. E. Lys-plasminogen is a significant intermediate in the activation of Glu-plasminogen during fibrinolysis in vitro. The Journal of Biological Chemistry 1992, S. 26150-26156]. Plasminogen (PLG) has a high binding affinity to endothelia cells and fibrin clots. The additional binding of tissue plasminogen (PLG) activator (tPA) leads to an activation and plasmin formation. The last mechanism is illustrated by a binding of plasminogen (PLG) on cell surface, which is activated by tPA to plasm in [Stricker, R. B.; Wong, Activation of plasminogen (PLG) by tissue plasminogen (PLG) activator on normal and thrombasthenic platelets: effects on surface proteins and platelet aggregation. Blood 1986, S. 275-280]. This therapy can be successful only, if the drug target plasminogen (PLG) is available sufficiently in the blood circulation and the thrombus site in a sufficient amount.

Plasminogen (PLG) is a zymogen, which, following partial cleavage by plasminogen (PLG) activators such as tissue-type plasminogen (PLG) activator [tPA] or urokinase plasminogen (PLG) activator [uPA], is converted to the proteolytically active form, plasmin [PM]. PM is a key protein of the fibrinolytic system, as it may degrade fibrin present in fibrin clots, the product of coagulation, to soluble fibrin degradation products/fragments leading to dissolution of the clot. The generation of plasmin occurs preferentially on the fibrin surface, which offers binding sites for plasminogen (PLG) and tPA. This binding stimulates plasminogen (PLG) activation, but also localizes the action of plasmin to sites of fibrin formation which promotes efficient clot lysis. The activated plasmin is a key enzyme in the fibrinolytic system. Thus, as long as plasmin is bound to fibrin clot matrix, it is not inhibited by the control inhibitor alpha-2-antiplasmin (A2AP), but released plasmin is instantaneously inhibited. Free plasmin has a very short half-life period in plasma of 0.1 sec. The half-life period of Glu-plasminogen and alpha-2-antiplasmin (A2AP) are 50 hours. In contrast Lys78-plasminogen (PLG) has a half-life period of only 20 hours [Fredenburgh, J. C.; Nesheim, M. E. Lys-plasminogen is a significant intermediate in the activation of Glu-plasminogen during fibrinolysis in vitro. The Journal of Biological Chemistry 1992, S. 26150-26156]. Plasmin exhibits preferential cleavage at the carboxyl side of Lysine and Arginine residues with higher selectivity than trypsin. It converts polymerized fibrin into soluble products [Castellino, Francis J.; Ploplis, Victoria A. Structure and function of the plasminogen (PLG)/plasmin system. Thrombosis and Haemostasis 2005, S. 647-654].

As used in the context of the present invention, the term “patient” may be understood in the broadest sense as any living being, which is preferably any animal, more preferably a mammal including human, in particular a human being. It will be understood that the plasminogen (PLG) is typically of the same species as the patient to be treated, in order to avoid undesired immunogenic side reactions. Optionally, the patient may also suffer from or may be at risk of developing a disorder selected from the group consisting of organ failure (e.g., organ failure of the kidney (e.g., acute kidney injury/failure (AKI), heart, lung, brain and veins), arterial obstructive disease, disorders in the microcirculation, disseminated intravascular coagulation (DIC), and a combination of two or more thereof in the final consequence of particular organ failure.

As used throughout the present invention, disseminated intravascular coagulation (DIC), may be microcirculation. Then, it may be also designated as disseminated intravascular microcoagulation.

The patient may or may not be suffering from a systemic disease. Throughout the present invention, the term “suffering from” as used herein may be understood in the broadest sense in a way that the patient has developed a pathological condition associated with disorder associated with a thrombotic event. This patient is preferably treated with plasminogen (PLG). Optionally, the patient may be suffering from a condition selected from the group consisting of organ failure, a thrombotic event, arterial obstructive disease, microcirculation, disseminated intravascular coagulation (DIC), and a combination of two or more thereof in particular organ failure, i.e., that such disorder is present in the patient. In a preferred embodiment, the term “suffering from” as used herein may be understood in the broadest sense in a way that the patient has developed a pathological condition associated with a thrombotic event. The patient suffering from a disorder not necessarily, but may optionally bear medicinal symptoms such as, e.g., one or more of the symptoms selected from the group consisting of acid-base disturbances (e.g., respiratory alkalosis or lactic acidosis), oliguria (even anuria), hyperglycemia, increased insulin requirements, tachypnea, hypocapnia, hypoxemia, liver dysfunction, hematologic abnormalities, azotemia, coagulation abnormalities, and ischemic colitis.

As indicated above, the present invention also refers to the prophylaxis of a thrombotic event. Such the patients risk to develop a (micro)thrombotic is preferably reduced with plasminogen (PLG). The term “being at risk of developing” means that the patient has an increased probability of obtaining a thrombotic event in comparison to the average probability throughout the population of the same species. More preferably, the risk is at least 5-fold increased, even more preferably the risk is at least 10-fold increased, even more preferably the risk is at least 100-fold increased, even more preferably the risk is at least 1000-fold increased.

In a preferred embodiment, the present invention relates to plasminogen (PLG) for use in a method for preventing or treating a thrombotic event in a patient, wherein said patient suffers from a disorder selected from the group consisting of organ failure, a thrombotic event, arterial obstructive disease, microcirculation, disseminated intravascular coagulation (DIC), and a combination of two or more thereof, in particular organ failure.

As used herein, a thrombotic event may be any formation of a blood clot inside a blood vessel. Such thrombotic event may obstruct the flow of blood through the circulatory system. This may lead to necrosis of tissue supplied by this blood vessel (also designatable as an infarction). In a preferred embodiment, a thrombotic event may be selected from the group consisting of deep vein thrombosis (DVT, e.g., in the legs, pelvic vein thrombosis), thrombosis in an embolism (e.g., a chronic or acute organ embolism (e.g., a pulmonary/lung embolism)), thrombosis in infarctions (e.g., myocardial infarctions, kidney, strokes, retinal vein occlusions), thrombosis in a disseminated intravascular coagulation (DIC), and thrombotic thrombocytopenic purpura (TTP).

In a preferred embodiment, a thrombotic event is an acquired thrombotic event. In a preferred embodiment, the thrombotic event is associated with a plasminogen (PLG) deficiency (e.g., an acquired plasminogen (PLG) deficiency, a temporary plasminogen (PLG) deficiency or a chronic plasminogen (PLG) deficiency). Such plasminogen (PLG) deficiency may optionally be caused due to a permanent consumption of plasminogen (PLG) in the blood flow (partially) closed atherosclerotic plaques or closed to stenosis of arteries or other blood vessels. Such plasminogen (PLG) deficiency can be simply observed either by measuring the activity of plasm in (after activation with e.g. urokinase) or by the immunometric detection of plasminogen (PLG).

Plasminogen (PLG) may be used as a direct treatment opportunity. The currently used tPA (tissue plasminogen (PLG) activator or uPA (Urokinase)) is used as an indirect therapy requiring at least a normal plasminogen (PLG) level. tPA may activate the plasminogen (PLG) to plasmin. This may be used to dissolve an already formed thrombus which may cause consecutive additional tissue damages. The low plasminogen (PLG) levels in those cases can be caused by the consumption of the plasminogen (PLG) protein, in particular Glu-plasminogen protein, in vivo. It was found that the patient with microthrombotic events often has a low concentration of plasminogen (PLG) in the plasma within the critical first 48 hours after the (micro)thrombotic event. After the injection of plasminogen (PLG), the total level of plasminogen (PLG) increased over a time period in a murine experimental setup. It is plausible that it may behave comparably in other mammals, including human. Additionally, not only the measurement of plasminogen (PLG) may be decisively but also the amount of alpha-2-antiplasmin. An increased amount of alpha-2-antiplasmin may inhibit the available plasminogen (PLG) molecules. Also, in this case, the injection of plasminogen (PLG) may balance the high concentration of alpha-2-antiplasmin and may lead to an improvement within the critical first 48 hours condition.

In a preferred embodiment, the thrombotic event is a thrombus of a kidney blood vessel having a diameter of less than 1 mm. In a preferred embodiment, the thrombotic event is a thrombus of a blood vessel having a diameter of less than 1 mm that is a kidney capillary. Accordingly, in a preferred embodiment, the present invention also relates to the plasminogen (PLG) for use in a method for preventing or treating microthrombosis in a patient's kidney.

In a preferred embodiment, the patient is at risk of developing microthrombi. In a preferred embodiment, the patient is at risk of developing microthrombi resulting in a thrombosis. In a preferred embodiment, the patient is at risk of developing microthrombi resulting in a thrombosis of the large blood vessels. In a preferred embodiment, the patient is at risk of developing microthrombi resulting in embolization. In a preferred embodiment, the patient is at risk of developing microthrombi resulting in embolization of the large blood vessels. In a preferred embodiment, the patient is at risk of developing microthrombi resulting in a thrombosis or embolization of the large blood vessels.

In a preferred embodiment, the patient is at risk of developing or is suffering from a pathological state selected from the group consisting of stenosis of arteria, veins, arterioles, venules, capillaries or from spasms in arteria, veins, arterioles, venules, capillaries resulting in diseases like lipoprotein(a)-anemia, iron deficiency, vitamin D deficiency, vitamin K deficiency, vitamin H deficiency, anemia, homocysteinaemia, protein Z deficiency, emboly, stroke, myocardial infarction, epistaxis, hypermenorrhea, Von Willebrand Syndrome, Morbus Meulengracht, a liver dysfunction, antiphospholipid-syndrome, migraine, a thyroid dysfunction, abortion, therapy failures in lysis therapy using activators for plasminogen (PLG) and a combination of two or more thereof.

In a preferred embodiment, the patient has a lower blood level of plasminogen (PLG), in particular Glu-plasminogen, than the blood level of plasminogen (PLG), in particular Glu-plasminogen, found throughout population of the same species.

Such lower blood level of plasminogen (PLG), in particular Glu-plasminogen, may be caused by any reason. In a preferred embodiment, the lower blood level of plasminogen (PLG), in particular Glu-plasminogen, is caused by one or more reasons selected from the group consisting of high physiologic or pathologic consumption of plasminogen (PLG), in particular Glu-plasminogen, a high elimination rate of plasminogen (PLG), a low expression rate of plasminogen (PLG), and the presence of high levels of one or more inhibitors of plasminogen (PLG), in particular Glu-plasminogen.

Optionally, the formation of microthrombi may be caused by a hyper-coagulability state present for a time which results in a consumption of plasminogen (PLG) (in particular plasminogen (PLG)) or may be caused by a hypo-fibrinolytic state caused by the continuous development of micro- and/or macro-thrombi or the present for both, optionally resulting in a consumption of plasminogen (PLG).

An inhibitor of plasmin(ogen) may be any chemical entity inhibiting the activity of plasm in(ogen) and/or decreasing the blood level of plasminogen (PLG). For example, an inhibitor of plasminogen (PLG) may be alpha-2-antiplasmin. This is further described in WO 2018/162754. In a preferred embodiment, the patient is characterized in that:
(a) the ratio of alpha-2-antiplasmin vs. plasminogen (PLG) (preferably plasminogen (PLG)) found in the blood of the patient is at least 1.1 fold higher in comparison to the average ratio found throughout a population of the same species; and/or
(b) the level plasminogen (PLG) (preferably plasminogen (PLG)) in the blood of the patient is at least 1% (mol/mol) lower in comparison to the average level found throughout population of the same species.

Preferably, the ratio of alpha-2-antiplasmin vs. plasminogen (PLG) (preferably plasminogen (PLG)) found in the blood of the patient is at least 1.15 fold higher, more preferably at least 1.2 fold higher, in particular at least 1.25 fold higher, in comparison to the average ratio found throughout population of the same species.

Preferably, the level plasminogen (PLG) (preferably plasminogen (PLG)) in the blood of the patient is at least 2% (mol/mol) lower, more preferably at least 5% (mol/mol) lower, in particular at least 10% (mol/mol), at least 20% (mol/mol), at least 30% (mol/mol), at least 40% (mol/mol), or at least 50% (mol/mol) lower, in comparison to the average level found throughout population of the same species.

In a preferred embodiment, the level of plasminogen (PLG) in the patient's blood is determined and, in case the determined level of plasminogen (PLG) is at least 10% (mol/mol)), at least 20% (mol/mol), at least 30% (mol/mol), at least 40% (mol/mol), or at least 50% (mol/mol) lower in comparison to the average level found throughout population of the same species, the patient is administered with a sufficient amount of plasminogen (PLG) to prevent or treat a thrombotic event.

The patient may or may not have any clinical symptoms. The patient may or may not have any ischemic regions. In a preferred embodiment, the patient suffers from at least one ischemic region. In a preferred embodiment, the patient suffers from at least one ischemic region that leads to necrosis of at least a part of a tissue without administration of plasminogen (PLG) to said patient.

In a preferred embodiment, plasminogen (PLG) is Glu-plasminogen and the patient suffers from at least one ischemic region that leads to necrosis of at least a part of a tissue without administration of Glu-plasminogen to said patient.

In this case, preferably, the administration of plasminogen (PLG) in accordance with the present invention may at least partly prevent or dissolve the thrombotic clot. In this case, preferably, the administration of plasminogen (PLG) in accordance with the present invention may at least partly prevent necrosis. Such necrosis due to inadequate blood supply to the affected area may also be designated as infarction.

An infarction may be understood in the broadest sense as tissue death (necrosis) due to inadequate blood supply to the affected area.

The thrombotic event may be caused by any reason. In a preferred embodiment, the thrombotic event is caused by an infarction, by hypercholesterolemia, or both. Also, the occlusion of the arterial way by thrombi can result in an infarction in the successional flow path in the depending organs (brain, heart, kidney, gastrointestinal tract, etc.). In a preferred embodiment, the thrombotic event is caused by an infarction, caused by any reason typical in the pathogenesis of infarctions, by the burst of an atherosclerotic plaque containing cholesterol crystals caused by a hypercholesterolemia, or both.

An infarction may be understood in the broadest sense as tissue death (necrosis) due to inadequate blood and therefore oxygen supply to the affected area. An infarction may be any infarction such as, e.g., a myocardial infarction, kidney infarction, stroke, etc.

In an alternative preferred embodiment, the thrombotic event is caused by at least one of the following: surgical intervention such as a vascular surgery (e.g., coronary bypass surgery), by an endoprosthesis (i.e., insertion of foreign bodies, e.g., blood vessel/vascular endoprosthesis), by an aneurysm, and by stenosis traumatic injuries. In an alternative preferred embodiment, the thrombotic event is associated with cancer, trauma, lack of movement, obesity, smoking, hormonal birth control, pregnancy and the period following birth, antiphospholipid syndrome, and certain genetic conditions. Genetic risk factors may include deficiencies of antithrombin, protein C, and protein S, and factor V Leiden mutation, and the administration of a systemic contraceptive, especially in combination with smoking, physical inactivity, especially prolonged lying in the sick, obesity, dehydration (exsiccosis), cancer, past thrombosis, pregnancy.

The patient may suffer from a single thrombotic event or from more than one thrombotic event. In a preferred embodiment, the patient suffers from more than one thrombotic event. In an alternative preferred embodiment, the patient suffers from a single thrombotic event.

Plasminogen (PLG) may be obtained from any source. It may be obtained from a commercial source or may be prepared by any means. For instance, it may be prepared as described in WO 2018/162754. It may be isolated from blood plasma and plasma fractions, in particular cryo-poor plasma, cryo-poor plasma reduced by one or two absorption steps (F IX/PCC or C1-Esterase Inhibitor), Paste or octanoic acid paste thereof as described in WO 2018/162754.

Plasminogen (PLG) may be administered to the patient by any means. In a preferred embodiment, plasminogen (PLG) is administered to the patient systemically. In a preferred embodiment, plasminogen (PLG) is administered to the patient by an administration route selected from the group consisting of intravenous (i.v.), intraarterial (i.a.), intracranial (i.c.), intraperitoneal (i.p.), intramuscular (i.m.), and subcutaneous (s.c.) injection), in particular by an administration route selected from the group consisting of intravenous (i.v.), intramuscular (i.m.), and subcutaneous (s.c.) injection). Alternatively or additionally, the pharmaceutical composition may also be suitable for other routes of administration such as, e.g., nasal or transdermal administration.

For administration, plasminogen (PLG) may be comprised in a pharmaceutical composition, i.e., is combined with least one pharmaceutically acceptable carrier. The terms “pharmaceutical composition” and “pharmaceutical formulation” may be understood interchangeably. As used herein, the terms “pharmaceutically acceptable carrier”, “pharmaceutically acceptable excipient”, “carrier” and “excipient” may be understood interchangeably in the broadest sense as any substance that may support the pharmacological acceptance of the plasminogen (PLG). Such pharmaceutical composition may be ready to use and may preferably be a liquid formulation, in particular an injection portion. The storage form may also be liquid, but may also be a dried form (e.g. a powder such as a powder comprising dried or freeze-dried plasminogen (PLG)) or may be a paste or syrup or the like. Optionally, a dried form, paste or syrup may be dissolved or emulsified prior to being administered to the patient.

A pharmaceutically acceptable carrier may exemplarily be selected from the list consisting of an aqueous buffer, saline, water, dimethyl sulfoxide (DMSO), ethanol, vegetable oil, paraffin oil or combinations of two or more thereof. Furthermore, the pharmaceutically acceptable carrier may optionally contain one or more detergent(s), one or more foaming agent(s) (e.g., sodium lauryl sulfate (SLS), sodium doceyl sulfate (SDS)), one or more coloring agent(s) (e.g., food coloring), one or more vitamin(s), one or more salt(s) (e.g., sodium, potassium, calcium, zinc salts), one or more humectant(s) (e.g., sorbitol, glycerol, mannitol, propylenglycol, polydextrose), one or more enzyme(s), one or more preserving agent(s) (e.g., benzoic acid, methylparaben, one or more antioxidant(s), one or more herbal and plant extract(s), one or more stabilizing agent(s), one or more chelating agents (e.g., ethylenediaminetetraacetic acid (EDTA), and/or one or more uptake mediator(s) (e.g., polyethylene imine (PEI), a cell-penetrating peptide (CPP), a protein transduction domain (PTD), an antimicrobial peptide, etc.).

The present invention also relates to a dosage unit of the pharmaceutical composition usable in the context of the treatment or prevention of the present invention. Exemplarily, the present invention may refer to a single dose container or to a multiple dosage form.

The dose range may be adjusted to the intended use. In a preferred embodiment, the patient is administered with plasminogen (PLG) at least once with a dose of plasminogen (PLG) in the range of 0.01 to 100 mg/kg body weight.

In a preferred embodiment, the patient is administered with plasminogen (PLG) at least once with a dose of plasminogen (PLG) in the range of 0.01 to 1 mg/kg body weight.

In a preferred embodiment, the patient is administered with plasminogen (PLG) at least once with a dose of plasminogen (PLG) in the range of 0.01 to 0.1 mg/kg body weight, 0.05 to 0.5 mg/kg body weight, 0.1 to 1 mg/kg body weight, 0.5 to 5 mg/kg body weight, 1 to 10 mg/kg body weight, or 1 to 100 mg/kg body weight.

In a preferred embodiment, a single dose administered intravenously is in the range of 0.01 to 1 mg/kg. Preferably, single dose administered intravenously is in the range of 0.02 to 0.9 mg/kg, 0.03 to 0.8 mg/kg, 0.04 to 0.7 mg/kg, 0.05 to 0.5 mg/kg, 0.1 to 0.4 mg/kg, or 0.25 to 0.35 mg/kg.

In a preferred embodiment, a single dose administered intramuscularly is in the range of 0.05 to 10 mg/kg. Preferably, single dose administered intramuscularly is in the range of 0.1 to 9 mg/kg, 0.2 to 8 mg/kg, 0.3 to 7 mg/kg, or 0.5 to 5 mg/kg.

In a preferred embodiment, a single dose administered subcutaneously is in the range of 0.1 to 100 mg/kg. Preferably, single dose administered subcutaneously is in the range of 0.5 to 90 mg/kg, 1 to 80 mg/kg, 2 to 70 mg/kg, or 5 to 50 mg/kg.

In a preferred embodiment, the patient is administered with plasminogen (PLG) at least once within 24 hours after the occurrence of a thrombotic event. In a preferred embodiment, the patient is administered with plasminogen (PLG) at least once within twelve hours or at least within six, five or four hours after the occurrence of a thrombotic event.

In an alternative preferred embodiment, the patient is administered within one week before being subjected to an event of high risk of developing a thrombotic event.

In a further alternative preferred embodiment, the patient is administered on a regular basis when being at risk of developing a thrombotic event.

As used herein, the term “on a regular basis” may be understood in the broadest sense as lasting for a time period of at least a week, at least a month, at least six months, or at least a year.

Such event of high risk of developing a thrombotic event may be any event that increases the risk of the patient for obtaining a thrombotic event. In a preferred embodiment, such event is a surgical intervention.

In this context, a surgical intervention may be any surgical intervention, preferably is vascular surgery. As used herein, vascular surgery may be understood in the broadest sense as being selected from bypass surgery (e.g., coronary bypass surgery), endoprosthesis (i.e., insertion of foreign bodies, e.g., blood vessel/vascular endoprosthesis), in aneurysms, stenosis traumatic injuries, and further vascular surgical interventions. In these cases, there may also be a risk for re-thrombosis caused by wound surfaces inside the blood vessel, by an endoprosthesis, etc. Those may activate the coagulation cascade resulting in a thrombus formation. To cover those risks after the activation of the coagulation cascade, the prophylactic administration of plasminogen (PLG) is surprisingly successful.

Further additional or alternative risk factors for developing a thrombotic event may include: cancer, trauma, lack of movement, obesity, smoking, hormonal birth control, pregnancy and the period following birth, antiphospholipid syndrome, and certain genetic conditions. Genetic risk factors may include deficiencies of antithrombin, protein C, and protein S, and factor V Leiden mutation. The underlying mechanism may involve some combination of decreased blood flow rate, increased tendency to clot, and injury to the blood vessel wall.

Still further additional or alternative risk factors for developing a thrombotic event may include, oral contraceptives (“pill”, especially here estrogen-reduced or estrogen-free), especially in combination with smoking, physical inactivity, especially prolonged lying in the sick, obesity, dehydration (exsiccosis), cancer, past thrombosis, pregnancy. Most commonly affected by deep vein thrombosis are the legs. One then speaks of upper or lower leg vein thrombosis. If both the calf, the popliteal fossa and the thigh are affected, it is called a multi-level thrombosis. A pelvic vein thrombosis is less common, but more dangerous because of the size of the vessel and the higher risk of pulmonary embolism. Pelvic vein thrombosis is feared in pregnant women, where a clot may dissolve after birth due to the lack of compression of the uterus and may lead to pulmonary embolism, which may be fatal. Another complication of thrombosis per se and DVT in particular is disseminated intravascular coagulation (DIC).

The patient may be administered with plasminogen (PLG) according to any administration scheme.

In a preferred embodiment, the patient is administered with plasminogen (PLG) once per day for a time period of three or more days. Preferably, the patient is administered with plasminogen (PLG) once per day for a time period of at least a week, for at least two weeks, at least four weeks, at least two months, or at least a year. In a preferred embodiment, the patient is administered is intravenously with plasminogen (PLG) once per day for a time period of three or more days, at least a week, for at least two weeks, at least four weeks, at least two months, or at least a year. In this context, a single dose may be a single dose in any dose range. In a preferred embodiment, administration of a single dose may be each in the range of 0.01 to 1 mg/kg body weight. Further preferred dose ranges are described herein.

In a preferred embodiment, the patient is administered with plasminogen (PLG) once per day for a time period of three or more days, wherein administration is intravenous administration, wherein intravenous administration of a single dose may be each in the range of 0.01 to 1 mg/kg body weight. Further preferred dose ranges are described herein.

In a preferred embodiment, the patient is administered with plasminogen (PLG) once per day for a time period of three or more days, preferably wherein administration is intraarterial administration, in particular intraarterial administration of a single dose each in the range of 0.01 to 1 mg/kg body weight. In a preferred embodiment, the patient is administered with plasminogen (PLG) once per day for a time period of three or more days, preferably wherein administration is intracranial administration, in particular intracranial administration of a single dose each in the range of 0.01 to 1 mg/kg body weight.

In a preferred embodiment, the patient is administered with plasminogen (PLG) once every two days for a time period of three or more days. Preferably, the patient is administered with plasminogen (PLG) every two days for a time period of at least a week, for at least two weeks, at least four weeks, at least two months, or at least a year. In a preferred embodiment, the patient is administered intramuscularly or intravenously, in particular intramuscularly, with plasminogen (PLG) once per day for a time period of three or more days, at least a week, for at least two weeks, at least four weeks, at least two months, or at least a year. In this context, a single dose may be a single dose in any dose range. In a preferred embodiment, administration of a single dose may be each in the range of 0.05 to 10 mg/kg body weight. Further preferred dose ranges are described herein. In a preferred embodiment, the patient is administered with plasminogen (PLG) once per day for a time period of three or more days, wherein administration is intramuscular administration, wherein intravenous administration of a single dose may be each in the range of 0.05 to 10 mg/kg body weight. Further preferred dose ranges are described herein.

In a preferred embodiment, the patient is administered with plasminogen (PLG) once every week for a time period of three or more weeks. Preferably, the patient is administered with plasminogen (PLG) once every week for a time period of at least two weeks, at least four weeks, at least two months, or at least a year. In a preferred embodiment, the patient is administered subcutaneously with plasminogen (PLG) once every week for a time period of three or more weeks, at least two weeks, at least four weeks, at least two months, or at least a year. In this context, a single dose may be a single dose in any dose range. In a preferred embodiment, administration of a single dose may be each in the range of 0.1 to 100 mg/kg body weight. Further preferred dose ranges are described herein. In a preferred embodiment, the patient is administered with plasminogen (PLG) once every week for a time period of three or more weeks, wherein administration is subcutaneous administration of a single dose in the range of 0.1 to 100 mg/kg body weight. Further preferred dose ranges are described herein. In a preferred embodiment, the patient is administered with a dose suitable to replace not more than 15% of the normal plasminogen (PLG) amount in the plasma compartment. Further preferred dose ranges are described herein. In a preferred embodiment, the patient is administered with a dose suitable to replace not more than 10% of the normal plasminogen (PLG) amount in the plasma compartment. Further preferred dose ranges are described herein. In a preferred embodiment, the patient is administered with a dose suitable to replace not more than 5% of the normal plasminogen (PLG) amount in the plasma compartment. Further preferred dose ranges are described herein.

In a preferred embodiment, the patient is administered once per day for a time period of three to seven days and is subsequently administered once every two days for a time period of three or more days. In an alternative preferred embodiment, the patient is administered once per day for a time period of three to seven days and is subsequently administered once per week for a time period of three or more weeks. In an alternative preferred embodiment, the patient is administered once per day for a time period of three to seven days and is subsequently administered once every two days for a time period of three or more days and is the, subsequently, administered once per week for a time period of three or more weeks.

Prior to administration, the level of plasminogen (PLG) in the patient's blood may be determined. In case the determined level of plasminogen (PLG) is at least 10% (mol/mol) lower in comparison to the average level found throughout population of the same species, the patient may be administered with a sufficient amount of plasminogen (PLG) to prevent or treat a thrombotic event.

Accordingly, in a preferred embodiment, the patient is:
(a) administered with plasminogen (PLG) at least once with a dose of plasminogen (PLG) in the range of 0.01 to 100 mg/kg body weight during the treatment period; and subsequently
(b) the level of plasminogen (PLG) in the patient's blood is determined in a step (i) and, in case the determined level of plasminogen (PLG) is at least 10% (mol/mol) lower in comparison to the average level found throughout population of the same species, the patient is administered with a sufficient amount of plasminogen (PLG) to prevent or treat a thrombotic event in a further step (ii), and optionally
(c) steps (i) and (ii) are conducted repeatedly as long as the level of plasminogen (PLG) determined in step (i) is at least 10% (mol/mol) lower in comparison to the average level found throughout population of the same species.

Further preferred dose ranges and treatment intervals are described herein.

A treatment period is the period of the duration of the respective treatment such as, e.g., a (micro)surgical intervention, and the time briefly afterwards such as, e.g., up to one day, up to two days, up to a week or up to a months after the respective treatment.

The patient may or may not suffer from a disease. In a preferred embodiment, the patient suffers from deep vein thrombosis, pelvic vein thrombosis, pulmonary embolism, an infarction of any organ, retinal vein occlusion, disseminated intravascular coagulation (DIC), thrombotic thrombocytopenic purpura (TTP), an angiopathy coincidence with thrombotic events in capillary flow path, in particular diabetic angiopathy, thrombophlebitis, or a combination of two or more thereof.

In a preferred embodiment, the patient suffers from disseminated intravascular coagulation (DIC), acute kidney/renal injury (AKI), sepsis, or a combination of two or more thereof.

In a preferred embodiment, the patient suffers from disseminated intravascular coagulation (DIC). In a preferred embodiment, the patient suffers from acute kidney/renal injury (AKI). In a preferred embodiment, the patient suffers from sepsis.

In a preferred embodiment, the patient is at risk of developing a thrombotic event in that said patient suffers from atherosclerosis or stenosis of arteries, has been subjected to or is subjected to a surgery, in particular vessel surgery (including reconstruction of a vessel (also known as vascular reconstruction) and/or bypass surgery), and/or has an endoprosthesis (e.g., blood vessel/vascular endoprosthesis). Bypass surgery may also include vessel transplantation (also known as vascular grafting). In a preferred embodiment, the patient suffering from an thrombotic event concomitantly suffers from or has previously suffered from atherosclerosis or stenosis of arteries, has been subjected to or is subjected to a surgery, in particular vessel surgery [including reconstruction of a vessel (also known as vascular reconstruction or vascular surgery) and/or bypass surgery], and/or has an endoprosthesis (e.g., blood vessel/vascular endoprosthesis).

The following examples and figures are intended to provide illustrative embodiments of the present invention described and claimed herein. These examples are not intended to provide any limitation on the scope of the invented subject-matter. The following figures, examples and claims further illustrate the invention.

### BRIEF DESCRIPTION OF THE FIGURES

FIG. 1 shows the reduction of glomerular filtration rate (GFR), depicted as percentage change over untreated mice, of mice administered with 10 mg/kg cholesterol (CC); mice administered with 10 mg/kg cholesterol and subsequently administered with 132 μL of a composition comprising 65 μg/mL of Glu-plasminogen 4 hours after cholesterol administration (CC+Glu-P); and mice administered with 132 μL of a composition comprising 65 μg/mL of Glu-plasminogen (PBS+Glu-P), each after 24 hours.

FIG. 2 shows the reduction of glomerular filtration rate (GFR), depicted as flow rate in μL/min, of untreated mice (Baseline); mice administered with 10 mg/kg cholesterol (CC); mice administered with 10 mg/kg cholesterol and subsequently administered with 132 μL of a composition comprising 65 μg/mL of Glu-plasminogen 4 hours after cholesterol administration (CC+Glu-P); and mice administered with 132 μL of a composition comprising 65 μg/mL of Glu-plasminogen (PBS+Glu-p), each after 24 hours.

FIG. 3 shows the infarct size, depicted as percentage of the whole kidney, of mice administered with 10 mg/kg cholesterol (CC); mice administered with 10 mg/kg cholesterol and subsequently administered with 132 μL of a composition comprising 65 μg/mL of Glu-plasminogen 4 hours after cholesterol administration (CC+Glu-P); and mice administered with 132 μL of a composition comprising 65 μg/mL of Glu-plasminogen (PBS+Glu-p), each after 24 hours.

FIG. 4 shows the percentage frequency of diagnosis among patients with a decreased plasminogen (PLG) level (n=600) having plasminogen (PLG) deficiency (black) and not having a plasminogen (PLG) deficiency (grey).

FIG. 5 shows alpha-2-antiplasmin (A2AP) activity in different patient groups (no disease/control population (Norm), lipoprotein(A) (LpA), iron deficiency (Iron), vitamin D deficiency (VitD), vitamin K deficiency (VitK), vitamin H deficiency (VitH), anemia (Anaem), homocysteine level (Hcys), protein Z deficiency (PZ), thrombosis (Thromb), embolism (Emb), stroke (Stroke), myocardial infarction (MI), epitaxies (Epist), hypermenorrhea (HM), von Willebrand syndrome (vWS), Morbus Meulengracht (Meul), liver diseases (Li), antiphospholipid diseases (APL), migraine (Migr), thyroid diseases (Thyr), abortions (Abort)).

FIG. 6 shows plasminogen (PLG) activity in different patient groups (the abbreviations are the same as used in FIG. 5).

FIG. 7 shows the (activity-based) ratio of alpha-2-antiplasmin:plasminogen (PLG) (A2AP/PLG) in different patient groups (the abbreviations are the same as used in FIG. 5).

FIG. 8 shows plasminogen (PLG) activity in a control population (CP) in comparison to a group of acute kidney injury/failure patients (AKI).

FIG. 9 shows alpha-2-antiplasmin (A2AP) activity in a control population (CP) in comparison to a group of acute kidney injury/failure patients (AKI).

FIG. 10 shows the (activity-based) ratio of alpha-2-antiplasmin:plasminogen (PLG) (A2AP/PLG) in a control population (CP) in comparison to a group of acute kidney injury/failure patients (AKI).

FIG. 11 shows plasminogen (PLG) activity in a control population (CP) in comparison to a group of disseminated intravascular coagulation patients (DIC).

FIG. 12 shows alpha-2-antiplasmin (A2AP) activity in a control population (CP) in comparison to a group of disseminated intravascular coagulation patients (DIC).

FIG. 13 shows the (activity-based) ratio of alpha-2-antiplasmin:plasminogen (PLG) (A2AP/PLG) in a control population (CP) in comparison to a group of disseminated intravascular coagulation patients (DIC).

FIG. 14 shows plasminogen (PLG) activity in a control population (CP) in comparison to a group of sepsis patients (Sepsis).

FIG. 15 shows alpha-2-antiplasmin (A2AP) activity in a control population (CP) in comparison to a group of sepsis patients (Sepsis).

FIG. 16 shows the (activity-based) ratio of alpha-2-antiplasmin:plasminogen (PLG) (A2AP/PLG) in a control population (CP) in comparison to a group of sepsis patients (Sepsis).

FIG. 17 shows the summary of a diagnostic study for acquired plasminogen (PLG) deficiency. Herein, the percentage plasminogen (PLG) activity is measured. Herein, 40% of patients with acute kidney/renal injury (AKI) and 62% of patients with disseminated intravascular coagulation patients (DIC) showed a statistically significant deficiency of plasminogen (PLG) in comparison to a control population (CP).

### EXAMPLES

Preparation of Glu-Plasminogen Preparations

Glu-plasminogen was prepared as described in Experimental Example 1 of WO 2018/162754 with a purity of >95% (w/w) based on the total protein content. The human Glu-plasminogen preparation contained 1256 μg/mL of Glu-plasminogen (as determined by enzyme-linked immunosorbent assay, ELISA).

The total protein content of said preparation was 1259 μg/mL (as determined by Bradford protein assay). Accordingly, the purity of Glu-plasminogen was found to be >99.7% by weight, based on total protein content. The high purity was also confirmed by sodium dodecyl sulfate-polyacrylamide gel electrophoresis (SDS-PAGE).

Proteolytic activity of the Glu-plasminogen (as determined by a standardized S-2288 (Chromogenix) proteolytic activity assay, referred to the total protein content, units/1.0 g/L of total protein content) was found below the detection limit.

The human Glu-plasminogen preparation contained only a negligible endotoxin level of <<1 EU/mL (as determined in a Limulus Amebocyte Lysate (LAL) endosafe endochrome assay according to European Pharmacopeia (version 5.0) chapter 2.6.14), and <0.35 g/L IgG, <0.05 g/L IgA and <0.35 g/L IgM (each determined in a nephelometric assay). Albumin (as determined by a polychromatic endpoint determination) and Lys-plasminogen (as determined by Western Blot) were not detectable.

In a further test of bioactivity, the concentration of the human Glu-plasminogen was set to 200 μg/mL and was then activated to plasmin. This corresponding to a concentration range naturally occurring in blood. The proteolytic activity of the obtained plasmin solution was determined by means of a para-nitrophenol-labeled (pNP-labeled) peptide substrate of plasmin. It was found that proteolytic plasmin activity was in a range of 109% in comparison to the activity naturally occurring in blood plasma plasmin proteolytic activity which was normalized to be 100%. Therefore, it was found that the human Glu-plasminogen was fully bioactive and could be converted in fully active plasmin.

### Example 1

Animal Model (Induction of Microthrombi in Mice by Cholesterol Crystals)

Triggering the Formation of Microthrombi in the Kidney

Mice were administered with cholesterol (CC) by means of an injection of 10 mg/kg, 100 μL/mouse in a blood vessel leading to the kidney. The time point of injection was considered as time point zero (0 hours). It was found that cholesterol leads to the formation of clots in smaller vessels in the kidney, in particular in kidney capillaries.

Treatment with Plasminogen (PLG)

Glu-plasminogen was prepared as described in Experimental Example 1 of WO 2018/162754 with a purity of >95% (w/w). The properties were those as described above. Some of the mice remained untreated. Those which were treated were administered with an intravenous (i.v.) injection of 132 μL/mouse of a composition containing 65 μg/mL Glu-plasminogen in phosphate buffered saline 4 hours after cholesterol administration. The Injection of phosphate buffered saline after 4 hours of cholesterol administration was used as a control group, with no treatment. As an additional control group, the injection of PBS instead of CC and the injection of 132 μL/mouse of a composition containing 65 μg/mL Glu-plasminogen in phosphate buffered saline was analyzed after 4 hours.

Readout

24 hours after cholesterol administration, the gglomerular filtration rate (GFR) was determined. Further, the infarct size in kidney was determined by means of staining with triphenyl tetrazolium chloride (TTC) of the kidney tissue. Further, the sacrificed mice were examined histologically, e.g., by means of determining the score the tubular injury (PAS), the endothelial injury (CD31) and the neutrophil immunocyte filtration.

Results and Discussion

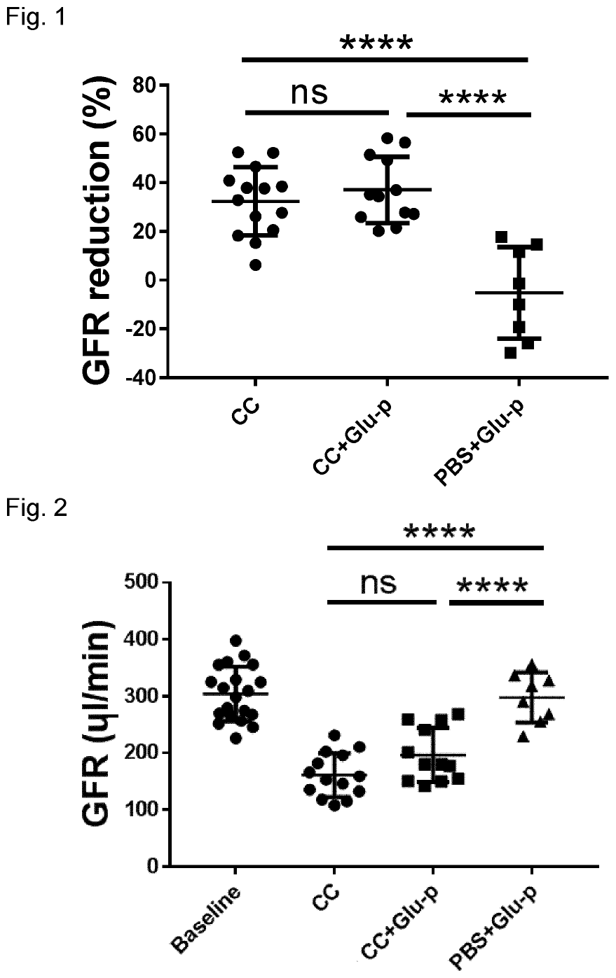

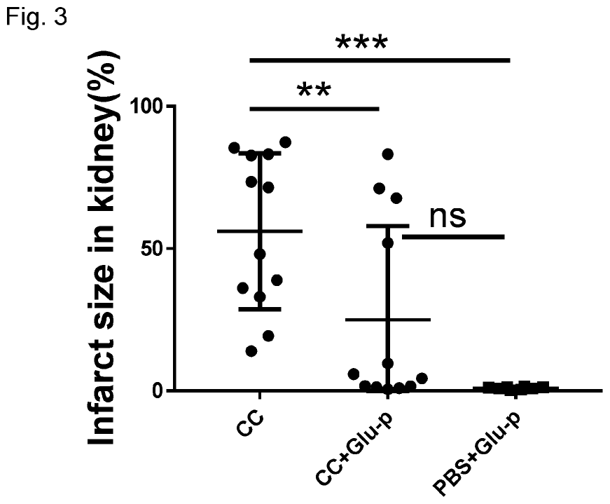

The quantitative results are depicted as FIGS. 1 to 3. Cholesterol administration was found to cause microthrombi. These were also found in histological observation of the mice kidney after 24 hours. These microthrombi were found to have a significant effect on the gglomerular filtration rate (GFR) (cf. FIGS. 1 and 2, samples including cholesterol (CC)) and causes necrosis of more than half (50%) of the kidney tissue (cf. FIG. 3, samples including only cholesterol (CC)). Administration of (Glu-)plasminogen alone was not found to have a significant impact on the gglomerular filtration rate (GFR) (cf. FIGS. 1 and 2, right). It does also not restore gglomerular filtration rate (GFR) effected by microthrombi caused by cholesterol administration (cf. FIGS. 1 and 2, samples including cholesterol (CC) and (Glu-)plasminogen (Glu-P).

However, administration of (Glu-)plasminogen effectively prevented necrosis of the tissue (cf. FIG. 3, samples including cholesterol (CC) and (Glu-)plasminogen (Glu-P).

Necrosis was reduced by the half in comparison to the necrosis found when only cholesterol is administered. Thus, (Glu-)plasminogen effectively reduced infarct size.

These results show that the administration of (Glu-)plasminogen effectively treats and prevents a patient suffering from (micro)thrombi.

The (Glu-)plasminogen produced according to this invention has surprisingly a high and excellent fibrinolytic activity in (micro)thrombotic events. Without being bound to this theory, it is assumed that (Glu-)plasminogen resolves existing microthrombi and can be used in the prophylaxis of micro- and/or macrothrombotic events. Such (micro)thrombotic events are often causal in infarctions such as, e.g., myocardial infarctions, strokes as well at kidney infarctions, a retinal vein occlusion, thrombotic thrombocytopenic purpura, etc.

### Example 2

Clinical Situations with an Acquired Plasminogen (PLG) Deficiency Caused by Plasminogen (PLG) Consumption.

In general, an acquired deficiency of plasminogen (PLG) could be expected in conditions causing an increased consumption of plasminogen (PLG), in particular Glu-plasminogen. This could be probably found in any kind of thrombotic events which have a longer history such as in atherosclerosis. The damage of the blood vessels' inside, the intima, could last for a longer time period. This will first lead to a permanent but slight activation of the coagulation system permanently converting fibrinogen into fibrin. As long as no clot occluding the whole blood vessels' diameter is formed, the blood flow is still intact. Such clot may permanently activate the fibrinolytic system. Here, plasminogen (PLG) (e.g., Glu-plasminogen) will be transformed into plasmin and, therefore, the plasminogen (PLG) (e.g., Glu-plasminogen) is consumed. Presently, there are two therapeutic approaches: first, until no occluding clot is formed, the inhibition of the coagulation such as, e.g., by vitamin K antagonists, is sufficient. When the clot is formed (which may result in, e.g., a myocardial infarction, a stroke etc.), until now, only thrombolytic therapy is an option. This is typically performed by activating plasminogen (PLG) by tPA, uPA or streptokinase.

The use of one of the three drugs requires the presence of the targeted enzyme plasminogen (PLG). In case of the described acquired plasminogen (PLG) deficiency, the target for the lysis therapy is, however, not present anymore. It was surprisingly found that in this case and in the any other acquired plasminogen (PLG) (e.g., Glu-plasminogen) deficiencies, the substitution of this protein may be supportive in the lifesaving treatment of those patients.

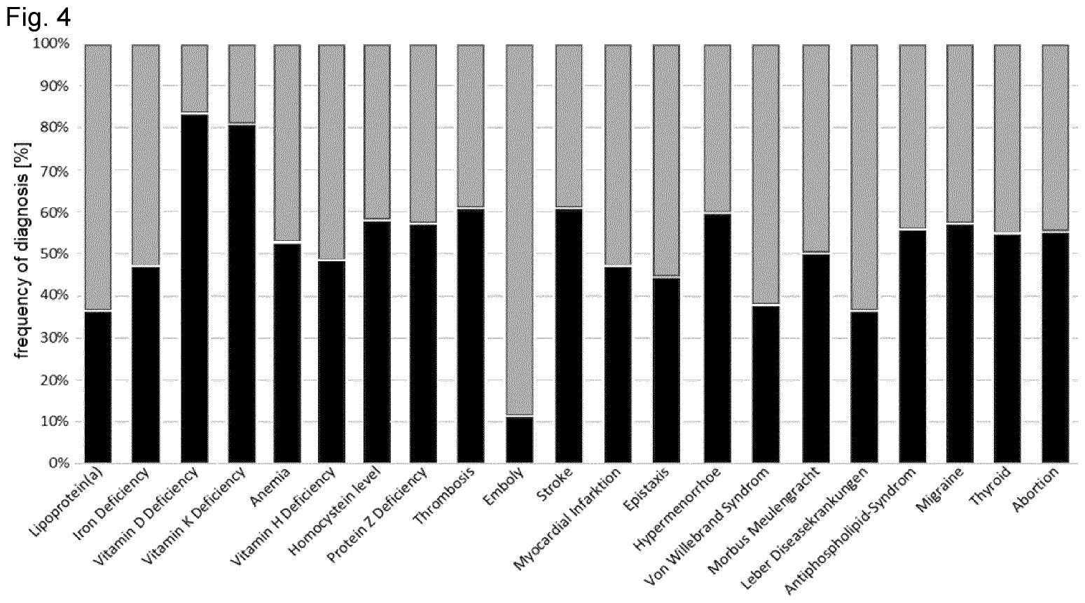

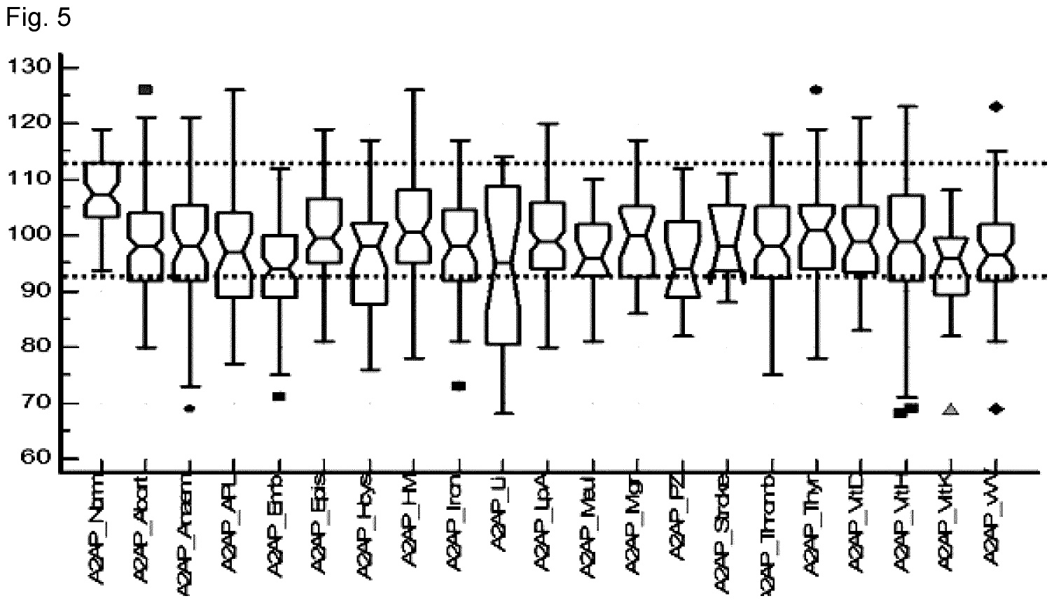

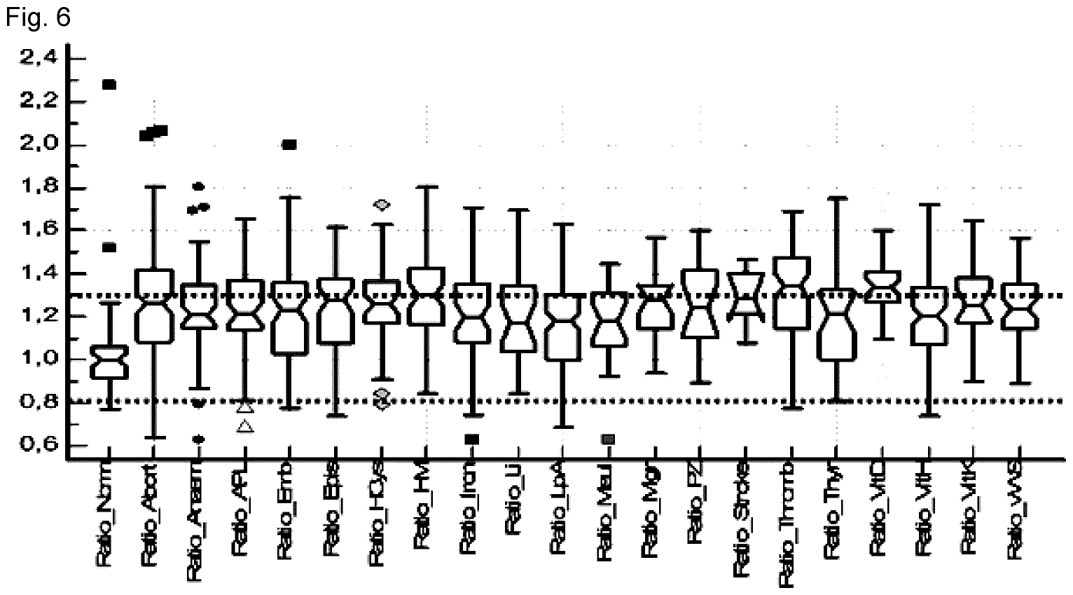

When thrombi are formed, plasminogen (PLG) (e.g., Glu-plasminogen) from plasma will be consumed due to the local activation. The higher the amount of fibrin located either in a single large macrothrombus or in many locations in microthrombi, especially during an event exacerbating during a longer time, the higher the consumption of plasminogen (PLG) typically is. This may result in transient acquired plasminogen (PLG) deficiency, tipping the balance to a hypofibrinolytic and thus prothrombotic state (cf. FIG. 4). Decreased plasminogen (PLG) levels have been shown in several conditions, including sepsis liver disease myocardial infarction, Argentine hemorrhagic fever, and after L-asparaginase therapy, thrombolytic therapy and surgery. Due to the protein's important role in fibrinolysis, a decreased plasminogen (PLG) level may compromise the body's ability to degrade fibrin and thus predispose it to thrombosis/thrombotic diseases.

Thus it was found that the cause of thrombotic disease may be either in hypercoagulation or in hypofibrinolysis. The current treatments focus on (hyper-) coagulation only by using heparin, warfarin, or F.X antagonists.

It was surprisingly found that the consumption of plasminogen (PLG) leads to a hypofibrinolysis which can be named also a transient acquired plasminogen (PLG) (e.g., Glu-plasminogen) deficiency. Thus, it was found that that the treatment of an acquired transient plasminogen (PLG) (e.g., Glu-plasminogen) deficiency is able to resolve thrombotic disease. Diagnostic studies in diseases with a high prevalence and a life-threatening state provided evidence for this finding.

### Example 2.1

Acute Kidney Injury/Failure (AKI)

The kidney is one of organs involved in multi organ failures also named “Multiple organ dysfunction syndrome (MODS)”.

This is defined as the presence of altered organ function in a patient who is acutely ill and in whom homeostasis cannot be maintained without intervention. MODS may eventually lead to multiple organ failure syndrome (MOFS) and death. Acute lung injury (ALI) and acute respiratory distress syndrome (ARDS) are common manifestations of MODS or MOFS. However, other conditions besides sepsis can cause MODS, including trauma, burns, and severe haemorrhagic shock. multiple organ dysfunctions, the extreme end of the continuum, are incremental degrees of physiologic derangements in individual organs (i.e., processes rather than events). Alteration in organ function can vary widely, ranging from a mild degree of organ dysfunction to frank organ failure (see multiple organ failure of sepsis, systemic inflammatory response syndrome (SIRS), toxic shock syndrome, and septic thrombophlebitis).

In an acute kidney failure, the microcirculation in the kidney is reduced in a way that no urine is produced any more. This allows an objective evaluation of the kidney function and their recovery.

In patients with an acute kidney injury/failure (AKI) and a pathogenesis over a longer time period, plasminogen (PLG) levels were found to be rather low in comparison to patients without a vascular genesis. A study focusing on this, was carried out together with Department of Clinical Chemistry (IKC) of the University Hospital Mannheim. Until March 2018, citrated plasma samples from 77 patients were collected, stored at below −40° C. All samples were measured batch-wise for plasminogen (PLG) and alpha-2-antiplasmin on a BCS XP Analyser (SIEMENS Healthcare) in a DIN EN ISO 15189 certified lab. A group of 53 healthy plasma donors were used as controls under the same conditions. The results were evaluated using u-test according to Mann-Whitney.

In the first validation study of patients with acute intrarenal kidney failure were selected at the Department of Clinical Chemistry (IKC), Mannheim, followed by criteria of acute kidney injury network (AKIN). Key parameters: creatinine raise up to 1.5-times the standard value in combination with the reduction of urine production.

Study-Outline Acute Kidney Injury/Failure (AKI)
Parameters: Alpha-2-antiplasmin (A2-AP), plasminogen (PLG), activity Ratio R=A2-AP/plasminogen (PLG) in blood samples, used for the standard diagnostic as well
77 patients with acute kidney injury/failure (AKI)
53 control population (healthy blood donors) (CP)

Results were analyzed by Mann-Whitney test and showed:
A significant (**) acquired plasminogen (PLG) deficiency in AKI patients
A significant (**) acquired alpha-2-antiplasmin deficiency in AKI patients
No significant difference of the (activity-based) ratio (A2AP/PLG)

TABLE 1
Summary of results of plasminogen (PLG)
activity in AKI patients versus control

| Table analyzed | Data |
|---|---|
| Column A | CP-plasminogen (PLG) (control |
| | group) |
| Column B | AKI-Plasminogen (PLG) (group of |
| | AKI patients) |
| Mann-Whitney test: | |
| P value (Gaussian approximation) | 0.0031 |
| P value summary | ** |
| Are medians significantly | Yes |
| different? (P < 0.05) | |
| One- or two-tailed P value? | Two-tailed |
| Sum of ranks in column A, B | 4096, 4420 |
| Mann-Whitney U | 1417 |

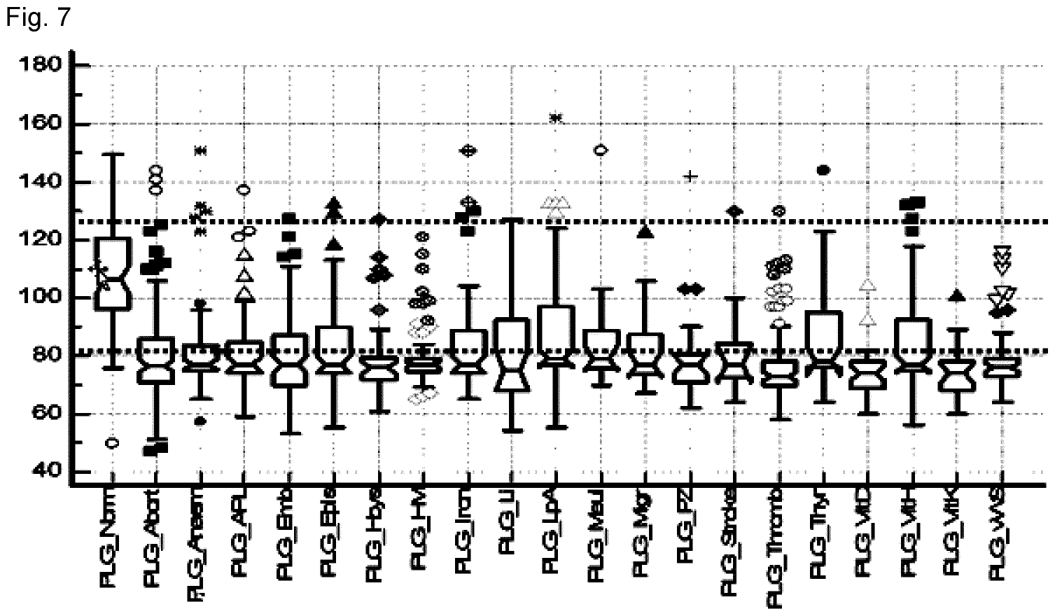

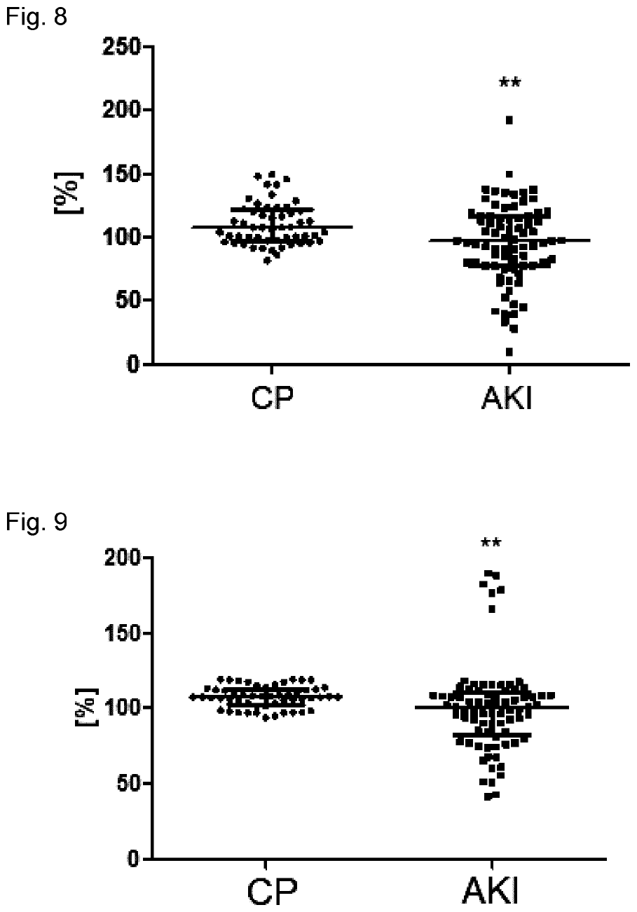

The Mann-Whitney test of acute kidney injury/failure (AKI) showed a significant difference of plasminogen (PLG) activity. P=0.0031 in patients (Ptx-77) and control group (CP-53). The results are shown in FIG. 8.

TABLE 2
Summary of results of alpha-2-antiplasmin activity
in AKI patients versus control group

| Table Analyzed | Data |
|---|---|
| Column A | CP-alpha-2-antiplasmin (control |
| | group) |
| Column B | AKI-alpha-2-antiplasmin (group of |
| | AKI patients) |
| Mann-Whitney test: | |
| P value (Gaussian approximation) | 0.0011 |
| P value summary | ** |
| Are medians significantly | Yes |
| different? (P < 0.05) | |
| One- or two-tailed P value? | Two-tailed |
| Sum of ranks in column A, B | 4160, 4355 |
| Mann-Whitney U | 1352 |

The Mann-Whitney test of acute kidney failure showed a significant difference of alpha-2-antiplasmin Activity. P=0.0011 in patients (Ptx-77) and control group (CP-53). The results are shown in FIG. 9.

TABLE 3
Summary of results of ratio of alpha-2-antiplasmin to
plasminogen (PLG) in AKI patients versus control group

| Table Analyzed | Data |
|---|---|
| Column A | CP-ratio (control group) |
| Column B | AKI-ratio (group of AKI patients) |
| Mann-Whitney test: | |
| P value (Gaussian approximation) | 0.1241 |
| P value summary | Ns |
| Are medians significantly | No |
| different? (P < 0.05) | |
| One- or two-tailed P value? | Two-tailed |
| Sum of ranks in column A, B | 3147, 5369 |
| Mann-Whitney U | 1716 |

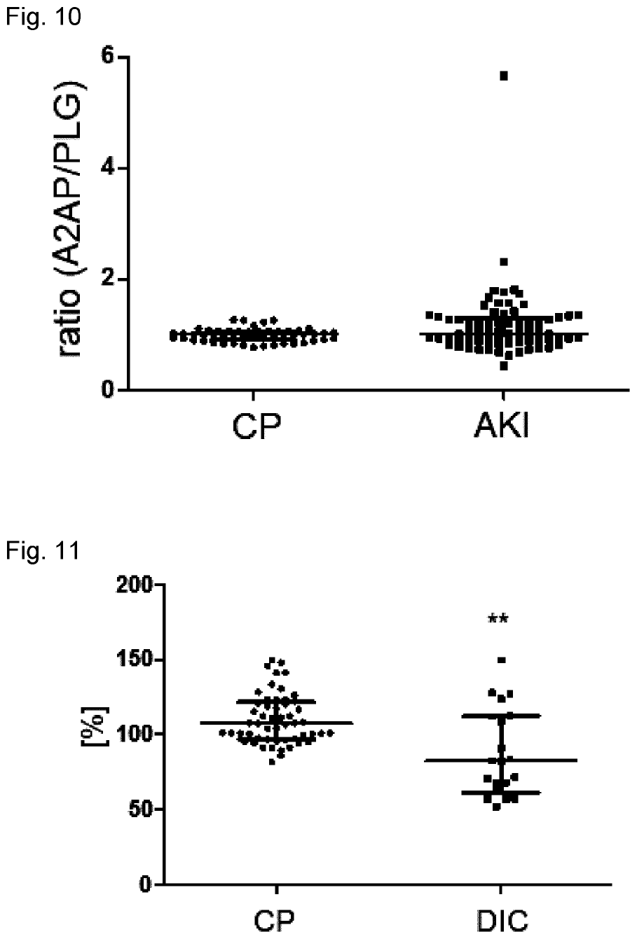

The Mann-Whitney test of acute kidney failure showed no significant difference of the (activity-based) ratio (A2AP/PLG; P=0.1241) in patients (Ptx-77) and in the control group (CP-53). The results are shown in FIG. 10.

### Example 2.2

Disseminated Intravascular Coagulation (DIC)

The validation study of patients with disseminated intravascular (micro-)coagulation (DIC) was carried out by the Department of Clinical Chemistry (IKC), Mannheim. Patients with DIC were identified by D-dimers levels and internal criteria.

Study-Outline-DIC:
Parameters: alpha-2-antiplasmin (A2-AP), plasminogen (PLG) activity. D-dimers, ratio R=A2-AP/plasminogen (PLG) in blood samples, used for the standard diagnostic as well
13 Patients with DIC (DIC)
53 control population (CP)

Results were analyzed by Mann-Whitney test and showed:
A significant (**) acquired plasminogen (PLG) deficiency in patients with DIC
No acquired alpha-2-antiplasmin deficiency in patients with DIC
A significant (***) difference of the (activity-based) ratio (A2AP/PLG)->increased inhibition of fibrinolysis

TABLE 4
Summary of results of plasminogen (PLG) activity
in DIC patients versus control group

| Table Analyzed | PLG |
|---|---|
| Column A | CP-plasminogen (PLG) (control |
| | group) |
| Column B | DIC-plasminogen (PLG) (group of |
| | DIC patients) |
| Mann-Whitney test | |
| P value (Gaussian approximation) | 0.0013 |
| P value summary | ** |
| Are medians significantly | Yes |
| different? (P < 0.05) | |
| One- or two-tailed P value? | Two-tailed |
| Sum of ranks in column A, B | 2256, 519 |
| Mann-Whitney U | 288.0 |

The Mann-Whitney test of DIC showed a significant difference of plasminogen (PLG) activity. P=0.0013 in patients (Ptx-13) and control group (CP-53). The results are depicted in FIG. 11.

TABLE 5
Summary of results of alpha-2-antiplasmin activity
in DIC patients versus control group

| Table Analyzed | A2-AP |
|---|---|
| Column A | CP-alpha-2-antiplasmin (control |
| | group) |
| Column B | DIC-alpha-2-antiplasmin (group of |
| | DIC patients) |
| Mann-Whitney test | |
| P value (Gaussian approximation) | 0.0730 |
| P value summary | ns |
| Are medians significantly | No |
| different? (P < 0.05) | |
| One- or two-tailed P value? | Two-tailed |
| Sum of ranks in column A, B | 2138, 637.5 |
| Mann-Whitney U | 406.5 |

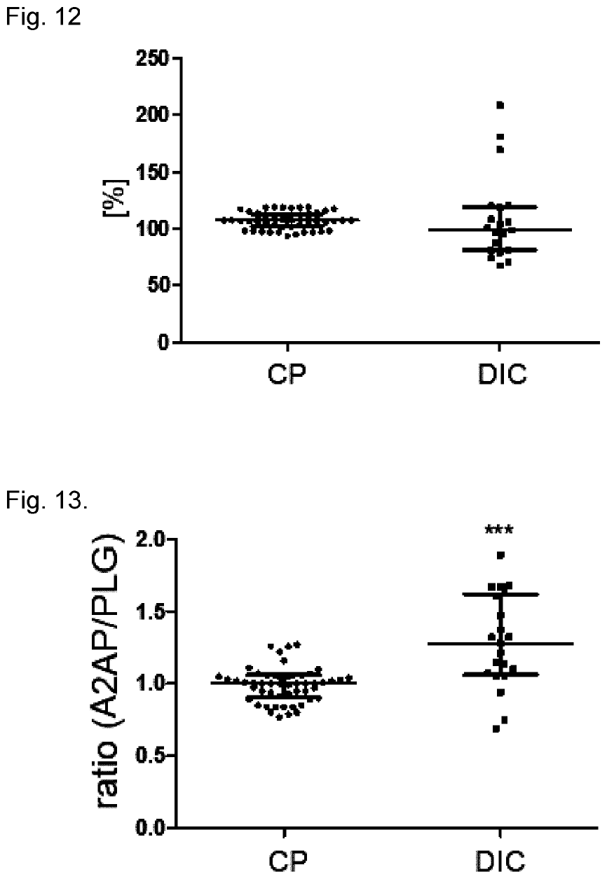

The Mann-Whitney test of DIC showed no significant difference of alpha-2-antiplasmin activity. P=0.073 in patients (Ptx-13) and control group (CP-53). The results are depicted in FIG. 12.

TABLE 6
Summary of results of ratio of alpha-2-antiplasmin to
plasminogen (PLG) in DIC patients versus control group

| Table Analyzed | Ratio-DIC |
|---|---|
| Column A | CP-ratio (control group) |
| Column B | DIC-ratio (group of DIC patients) |
| Mann-Whitney test | |
| P value (Gaussian approximation) | <0.0001 |
| P value summary | *** |
| Are medians significantly | Yes |
| different? (P < 0.05) | |
| One- or two-tailed P value? | Two-tailed |
| Sum of ranks in column A, B | 1634, 1141 |
| Mann-Whitney U | 203.0 |

The Mann-Whitney test of DIC showed a significant increased (activity-based) (activity-based) ratio (A2AP/PLG; P<0.0001) in patients (Ptx-13) than in the control group (CP-53). The results are depicted in FIG. 13.

In summary, in a study of 53 individuals of a (healthy) control population (CP), 77 patients with acute kidney/renal injury (AKI) and 21 patients with disseminated intravascular coagulation patients (DIC), 40% of patients with AKI and 62% of patients with DIC showed a statistically significant deficiency of plasminogen (PLG) in comparison to the control population (CP). Herein, the Mann-Whitney test showed a median interquartile range p>0.003 for CP vs. AKI and >0.001 for CP vs. DIC. The results are depicted in FIG. 17.

### Example 2.3

Sepsis

The validation study of patients with sepsis was carried out at the Department of Clinical Chemistry (IKC), Mannheim (identification by D-dimers and internal criteria).

Study-Outline-Sepsis:
Parameters: alpha-2-antiplasmin (A2AP), plasminogen (PLG) activity, Ratio R=A2-AP/plasminogen (PLG), PCTP, DD in blood samples, used for the standard diagnostic as well.
9 sepsis patients (sepsis)
53 control population (CP)

Results were analyzed by Mann-Whitney test and showed:
A significant (*) acquired Plasminogen (PLG) deficiency in sepsis patients
No acquired alpha-2-antiplasmin deficiency in patients with sepsis
No significant difference of the (activity-based) ratio (A2AP/PLG)

TABLE 7
Summary of results of plasminogen (PLG) activity
in sepsis patients versus control group

| Table Analyzed | Sepsis-PLG |
|---|---|
| Column A | CP-plasminogen (PLG) (control |
| | group) |
| Column B | Sepsis-plasminogen (PLG) (group |
| | of sepsis patients) |
| Mann-Whitney test | |
| P value (Gaussian approximation) | 0.0377 |
| P value summary | * |
| Are medians significantly | Yes |
| different? (P < 0.05) | |
| One- or two-tailed P value? | Two-tailed |
| Sum of ranks in column A, B | 1774, 179 |
| Mann-Whitney U | 134.0 |

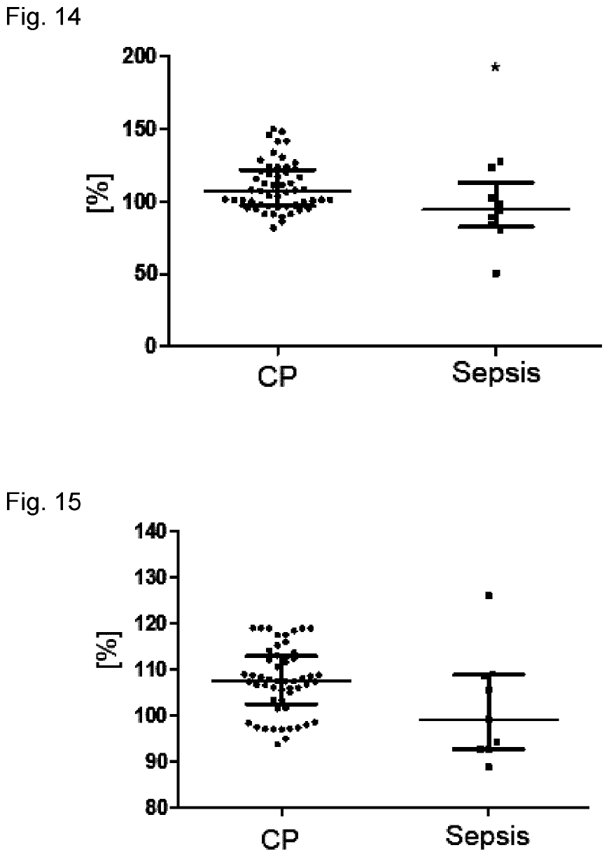

The Mann-Whitney test showed a significant difference of plasminogen (PLG) activity. P=0.0377 in patients (Ptx-9) and control group (CP-53). The results are depicted in FIG. 14.

TABLE 8
Summary of results of alpha-2-antiplasmin activity
in sepsis patients versus control group

| Table Analyzed | Sepsis-A2AP |
|---|---|
| Column A | CP-alpha-2-antiplasmin (control |
| | group) |
| Column B | Sepsis-Alpha-2-antiplasimin (group |
| | of sepsis patients) |
| Mann-Whitney test | |
| P value (Gaussian approximation) | 0.0704 |
| P value summary | ns |
| Are medians significantly | No |
| different? (P < 0.05) | |
| One- or two-tailed P value? | Two-tailed |
| Sum of ranks in column A, B | 1761, 192.5 |
| Mann-Whitney U | 147.5 |

The Mann-Whitney test showed no significant difference of alpha-2-antiplasmin activity: P=0.0704 in patients (Ptx-9) and control group (CP-53). The results are depicted in FIG. 15.

TABLE 9
Summary of results of ratio of alpha-2-antiplasmin to plasminogen
(PLG) in sepsis patients versus control group

| Table Analyzed | Sepsis-Ratio |
|---|---|
| Column A | CP-ratio (control group) |
| Column B | Sepsis-ratio (group of sepsis patients) |
| Mann-Whitney test | |
| P value (Gaussian approximation) | 0.2182 |
| P value summary | ns |
| Are medians significantly | No |
| different? (P < 0.05) | |
| One- or two-tailed P value? | Two-tailed |
| Sum of ranks in column A, B | 1608, 345.5 |
| Mann-Whitney U | 176.5 |

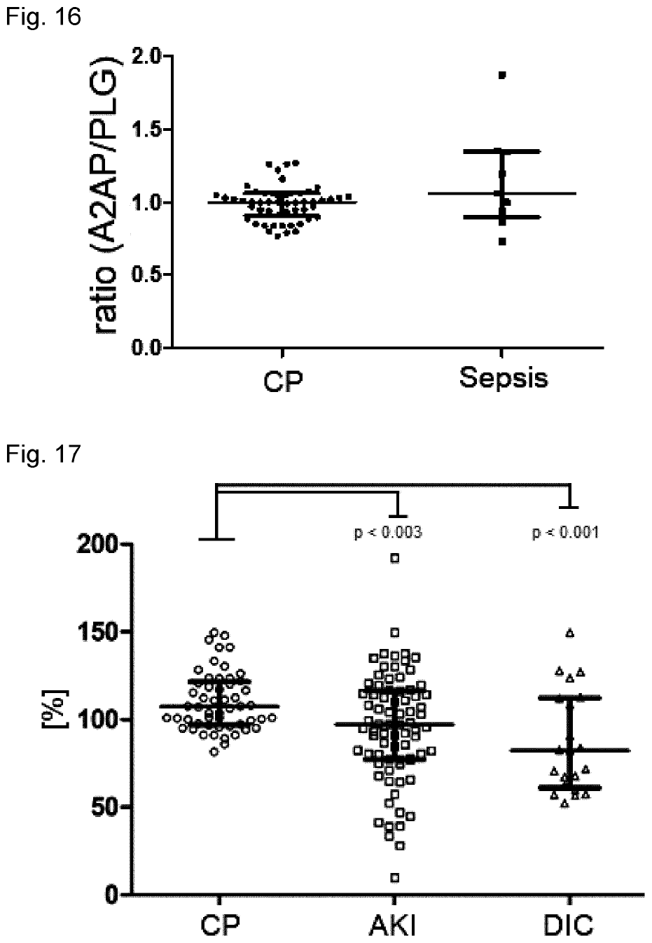

The Mann-Whitney test showed a non-significant but increased (activity-based) ratio (A2AP/PLG; P=0.2182) in patients (Ptx-13) than in the control group (CP-53). The results are depicted in FIG. 16.

### Example 2.4

Control Population

Raw-data: measurement of plasminogen (PLG) and A2AP in the control population (CP), healthy plasma donors from a plasma center.

Reference ranges could be defined in the control population PLG: 90% to 144%; A2AP: 97% to 119%; and Ratio: 0.80 to 1.25.

TABLE 10
Normalized activity of plasminogen (PLG) and A2AP of
controls (normalized to an average activity of 100%)

| Controls | No. | PLG | A2AP | Ratio |
|---|---|---|---|---|
| | 1 | 101.3 | 105.1 | 1.04 |
| 5 of 50 individuals => 1.26 | 2 | 94.4 | 97.3 | 1.03 |
| | 3 | 107.3 | 97.1 | 0.90 |
| | 4 | 103.7 | 107.3 | 1.03 |
| | 5 | 103.8 | 108.9 | 1.05 |
| | 6 | 100.4 | 105.9 | 1.05 |
| | 7 | 96.3 | 101.4 | 1.05 |
| | 8 | 81.6 | 103.3 | 1.27 |
| | 9 | 95 | 94.9 | 1.00 |
| | 10 | 147.9 | 114.1 | 0.77 |
| | 11 | 120.8 | 113 | 0.94 |
| | 12 | 122 | 108.3 | 0.89 |
| | 13 | 116.5 | 98.5 | 0.85 |
| | 14 | 99.6 | 110.5 | 1.11 |
| | 15 | 100.9 | 98.3 | 0.97 |
| | 16 | 120.1 | 101.5 | 0.85 |
| | 17 | 107.6 | 108.7 | 1.01 |
| | 18 | 112.4 | 118.9 | 1.06 |
| | 19 | 96.7 | 106 | 1.10 |
| | 20 | 123.4 | 117.4 | 0.95 |
| | 21 | 100.9 | 107.4 | 1.06 |
| | 22 | 91.3 | 97 | 1.06 |
| | 23 | 123.4 | 117.4 | 0.95 |
| | 24 | 100.9 | 107.4 | 1.06 |
| | 25 | 91.3 | 97 | 1.06 |
| | 26 | 141.1 | 112 | 0.79 |
| | 27 | 110.9 | 108 | 0.97 |
| | 28 | 145.5 | 115.9 | 0.80 |
| | 29 | 100.1 | 106.7 | 1.07 |
| | 30 | 100.7 | 107.3 | 1.07 |
| | 31 | 149.5 | 118.9 | 0.80 |
| | 32 | 130.4 | 118.9 | 0.91 |
| | 33 | 95.7 | 97.9 | 1.02 |
| | 34 | 108 | 107.8 | 1.00 |
| | 35 | 91.1 | 115.2 | 1.26 |
| | 36 | 141.3 | 118.9 | 0.84 |
| | 37 | 111.3 | 105.6 | 0.95 |
| | 38 | 126.3 | 106.5 | 0.84 |
| | 39 | 97.2 | 112.6 | 1.16 |
| | 40 | 112.3 | 112.3 | 1.00 |
| | 41 | 89.3 | 108.8 | 1.22 |
| | 42 | 97.5 | 97.4 | 1.00 |
| | 43 | 107.6 | 106 | 0.99 |
| | 44 | 95.1 | 96.9 | 1.02 |
| | 45 | 117.3 | 118.9 | 1.01 |
| | 46 | 94 | 93.7 | 1.00 |
| | 47 | 128.2 | 113.7 | 0.89 |
| | 48 | 85.9 | 108.5 | 1.26 |
| | 49 | 106.4 | 106.7 | 1.00 |
| | 50 | 123.7 | 103.4 | 0.84 |
| | 51 | 133.4 | 111.5 | 0.84 |
| | 52 | 115.4 | 107.4 | 0.93 |
| | 53 | 118.6 | 118.3 | 1.00 |
| Mean | | 110.06 | 107.52 | 0.99 |
| Standard Dev | | 16.65 | 7.19 | 0.12 |
| Min | | 81.60 | 93.70 | 0.77 |
| Max | | 149.50 | 118.90 | 1.27 |
| Range | | 67.90 | 25.20 | 0.49 |
| MW + 2SD | | 143.36 | 121.89 | 1.23 |
| MW − 2SD | | 76.76 | 93.14 | 0.75 |
| VK | | 15.13% | 6.69% | 11.95% |
| 80% percentile | | <89.57 | <96.915 | <0.80 |
| 120% percentile | | >144.84 | >118.9 | >1.25 |

### Example 2.5

Diagnosis in Patients with Acquired Plasminogen (PLG) Deficiencies Caused by Plasminogen (PLG) Consumption

At Centrum für Blutgerinnungsstörungen and Transfusionsmedizin (CBT) Bonn, from a total of 6000 (from non-acute states from GP's offices (general practitioners' medical offices/family practices)) patients with measured plasminogen (PLG) levels, all levels with decreased plasminogen (PLG) levels were selected and analyzed (n=700 {11.6%}). The frequency of diagnosis among patients with a decreased plasminogen (PLG) level was analyzed. Many diagnostic hits occurred where a plasminogen (PLG) deficiency was not expectable. The results are shown in FIG. 4 and Table 11. A prospective study at the CBT is carried out to prove these findings. In parallel, a prospective study in acute patients with the same diagnosis is carried out as well.

TABLE 11
Results of diagnosis in different patient groups, correlations of different parameters

| | | | | | | | | | | | | | |
|---|---|---|---|---|---|---|---|---|---|---|---|---|---|
| | | Thrombl | | Prot C | APC | | | | | PAP | | | |
| PLG | Quick | n Time | APTT | Act | Ratio | ATIII | AZAP | Ratio | PAI 1 | Complex | D-Dimer | PLT | CRP |

| | | | | | | | | | | | | | |
|---|---|---|---|---|---|---|---|---|---|---|---|---|---|
| PLG | 0.1007 | −0.1078 | −0.0032 | 0.2576 | −0.0103 | 0.1813 | 0.2097 | −0.8148 | 0.1018 | −0.0949 | −0.0035 | 0.2000 | 0.1793 |
| Quick | | −0.0905 | −0.2004 | 0.6040 | 0.1218 | 0.1539 | 0.2505 | 0.0537 | −0.0438 | −0.0387 | 0.0298 | 0.0357 | −0.0497 |
| Thrombl | | | 0.0839 | −0.0831 | 0.0635 | −0.0483 | −0.0368 | 0.0802 | −0.0251 | −0.1529 | −0.0288 | 0.4293 | −0.0754 |
| n Time | | | | | | | | | | | | | |
| APTT | | | | −0.0864 | −0.0025 | −0.0559 | −0.0167 | −0.0063 | 0.0409 | −0.1795 | −0.0618 | 0.0515 | 0.0204 |
| Prot C | | | | | −0.0104 | 0.2444 | 0.3517 | −0.0354 | 0.0922 | −0.1784 | −0.0185 | 0.0630 | 0.0373 |
| Act. | | | | | | | | | | | | | |
| APC | | | | | | −0.0548 | 0.0509 | 0.0444 | −0.0409 | −0.3889 | −0.0500 | −0.0498 | −0.0355 |
| Ratio | | | | | | | | | | | | | |
| ATIII | | | | | | | 0.4439 | 0.0512 | −0.0878 | −0.0624 | −0.1363 | 0.2074 | −0.1637 |
| AZAP | | | | | | | | 0.3335 | 0.0394 | 0.0469 | −0.1000 | 0.2259 | −0.0136 |
| Ratio | | | | | | | | | −0.0721 | 0.1070 | −0.0373 | −0.0378 | −0.1433 |
| PAI 1 | | | | | | | | | | −0.2503 | 0.0517 | −0.0330 | 0.1750 |
| PAP | | | | | | | | | | | −0.0218 | −0.2029 | 0.1150 |
| Complex | | | | | | | | | | | | | |
| D-Dimer | | | | | | | | | | | | −0.0869 | 0.2367 |
| PLT | | | | | | | | | | | | | 0.0359 |

Table 11 describes the correlation coefficient from a linear regression R²=r. This was performed in the computer program Microsoft Excel (using the function “KORREL” in the German version) by correlating each two parameters with each other. This indicates whether there is a statistical relationship between two parameters or not. It was assumed that a correlation rates r of −0.5>r<0.5 indicate no or a weak correlation. Correlation rates r of −1.0 to −0.5 or 0.5 to 1.0 indicate a significant correlation. Thus, there was only a significant correlation for PLG with Ratio and for Quick with Pro C Act.

In Table 11, the following parameters are shown: PLG: plasminogen [IU/mL]; Quick [sec]; thrombin time [sec]; APTT: activated partial thromboplastine time [sec]; Prot C Act=protein C-activated [IU/mL]; APC ratio: activated protein C resistance [%]; ATIII=antithrombin III [%]; A2AP: alpha-2-antiplasmin [%]; ratio (A2AP/PLG), PAI-1: plasminogen activator inhibitor-1 [ng/mL], PAP complex: plasm iniogen-antiplasm in.complex; D-Dimer: D-dimer; PLT=platelets [×10⁶/μL]; CRP: C-reactive protein [μg/mL]

TABLE 12
Results of p-values in different patient groups

| Disease | Abbreviation | P-value | n |
|---|---|---|---|
| Lipoprotein(A) | LpA | <0.0001 | 119 |
| Iron deficiency | Iron | <0.0001 | 55 |
| Vitamin D deficiency | VitD | <0.0001 | 37 |
| Vitamin K deficiency | VitK | <0.0001 | 21 |
| Anemia | Anaem | <0.0001 | 56 |
| Homocysteine level | Hcys | <0.0001 | 45 |
| Protein Z deficiency | PZ | <0.0001 | 21 |
| Thrombosis | Thromb | <0.0001 | 81 |
| Embolism | Emb | <0.0001 | 86 |
| Stroke | Stroke | <0.0005 | 9 |
| Vitamin H deficiency | VitH | <0.0001 | 87 |
| Myocardial infarction | MI | n.s. | 3 |
| Epitaxis | Epist | <0.0001 | 71 |
| Hypermenorrhoe | HM | <0.0001 | 61 |
| Von Willebrand syndrom | vWS | <0.0001 | 54 |
| Morbus Meulengracht | Meul | <0.0001 | 21 |
| Liver diseases | Li | <0.0001 | 29 |
| Antiphospholipid diseases | APL | <0.0001 | 51 |
| Migraine | Migr | <0.0001 | 29 |
| Thyroid diseases | Thyr | <0.0001 | 41 |
| Abortions | Abort | <0.0001 | 158 |
| No/control population | Norm | n/A | 55 |

In this study, plasminogen (PLG) was identified as an independent parameter. There was no correlation to any other parameter from the coagulation or fibrinolytic system except the weak correlation to the (activity-based) ratio (A2AP/PLG) (where plasminogen (PLG) is present. All samples were drawn from non-acute patients. Therefore, only a few patients with acute myocardial infarction were present in this study. The selected patient population (with low plasminogen (PLG) levels) showed a with normal A2AP levels.

1-30. (canceled)

31. A method for preventing or treating a thrombotic event in a patient, wherein the patient is administered with a sufficient amount of plasminogen, and wherein the patient is at risk of developing or is suffering from microthrombi having diameters of less than 1 mm.

32. The method of claim 31, wherein the plasminogen is Glu-plasminogen.

33. The method of claim 32, wherein the patient bears an acquired Glu-plasminogen deficiency caused by increased Glu-plasminogen consumption, decreased biosynthesis of Glu-plasminogen, or a combination of both.

34. The method of claim 31, wherein the plasminogen is Glu-plasminogen and the patient suffers from at least one ischemic region that would cause necrosis of at least a part of a tissue without administration of the Glu-plasminogen to the patient.

35. The method of claim 31, wherein the plasminogen has no proteolytic activity.

36. The method of claim 31, wherein the patient bears an acquired plasminogen deficiency.

37. The method of claim 36, wherein the acquired plasminogen deficiency is caused by increased plasminogen consumption.

38. The method of claim 31, wherein the plasminogen is Lys-plasminogen or a combination of Glu-plasminogen and Lys-plasminogen or a combination of Glu-plasminogen and Lys-plasminogen and one or more other plasminogen derivatives.

39. The method of claim 31, wherein the patient is at risk of developing or is suffering from microthrombi resulting in a thrombosis or embolization of the large blood vessels.

40. The method of claim 31, wherein the patient is at risk of developing or is suffering from a pathological state selected from the group consisting of stenosis of arteria, veins, arterioles, venules, capillaries or from spasms in arteria, veins, arterioles, venules, capillaries resulting in diseases like lipoprotein(a)-anemia, iron deficiency, vitamin D deficiency, vitamin K deficiency, vitamin H deficiency, anemia, homocysteinaemia, protein Z deficiency, emboly, stroke, myocardial infarction, epistaxis, hypermenorrhea, Von Willebrand Syndrome, Morbus Meulengracht, a liver dysfunction, antiphospholipid-syndrome, migraine, a thyroid dysfunction, abortion, therapy failures in lysis therapy using activators for plasminogen and a combination of two or more thereof.

41. The method of claim 31, wherein the patient has a lower blood level of plasminogen than the average blood level of plasminogen found throughout a population of the same species.

42. The method of claim 41, wherein the lower blood level of plasminogen is caused by one or more reasons selected from the group consisting of high physiologic or pathologic consumption of plasminogen, a high elimination rate of plasminogen, a low expression rate of plasminogen, and the presence of high levels of one or more inhibitors of plasminogen.

43. The method of claim 42, wherein the lower blood level of plasminogen is caused by high physiologic or pathologic consumption of plasminogen.

44. The method of claim 31, wherein the level of plasminogen in the patient's blood is determined and, the patient is administered with a sufficient amount of plasminogen to prevent or treat a thrombotic event if the determined level of plasminogen is at least 10% (mol/mol) lower in comparison to the average level of plasminogen found throughout population of the same species.

45. The method of claim 31, wherein the microthrombi are microthrombi of capillaries.

46. The method of claim 31, wherein the patient suffers from at least one ischemic region that would cause necrosis of at least a part of a tissue without administration of plasminogen to the patient.

47. The method of claim 31, wherein the patient suffers from more than one thrombotic event.

48. The method of claim 31, wherein the patient suffers from at least one ischemic region that would cause necrosis of at least a part of a tissue without the administration of plasminogen to the patient, and at least one thrombotic event.

49. The method of claim 31, wherein the thrombotic event is caused by an infarction or wherein the thrombotic event results in an infarction.

50. The method of claim 31, wherein the thrombotic event is caused by the burst of an atherosclerotic plaque containing cholesterol crystals caused by hypercholesterolemia.

51. The method of claim 31, wherein the thrombotic event is caused by an infarction, and by the burst of an atherosclerotic plaque containing cholesterol crystals caused by hypercholesterolemia.

52. The method of claim 31, wherein the thrombotic event causes an infarction.

53. The method of claim 31, wherein the patient is administered with the plasminogen at least once with a dose of plasminogen in the range of 0.01 to 100 mg/kg body weight or in the range of 0.01 to 1 mg/kg body weight.

54. The method of claim 31, wherein the patient is administered with the plasminogen at least once within 24 hours after the occurrence of a thrombotic event, within one week before being subjected to an event with a high risk of developing a thrombotic event or on a regular basis when the patient is at risk of developing a thrombotic event.

55. The method of claim 54, wherein the event with a high risk of developing a thrombotic event is a surgery.

56. The method of claim 31, wherein the patient is administered with the plasminogen according to one of the following administration schemes:
(A) the patient is administered the plasminogen intravenously once per day for at least three days;
(B) the patient is administered the plasminogen intraarterially once per day for at least three days;
(C) the patient is administered the plasminogen intracranially once per day for at least three days;
(D) the patient is administered the plasminogen intramuscularly once every two days for at least three days;
(E) the patient is administered with plasminogen subcutaneously once every week for at least three weeks; or
(F) the patient is administered with plasminogen once per day for three to seven days and is subsequently administered once every two days for at least three days or once per week for at least three weeks.

57. The method of claim 31, wherein the patient is administered with a dose of the plasminogen suitable to replace not more than 15%, not more than 10%, or not more than 5%, of the normal plasminogen amount in the plasma compartment of the blood.

58. The method of claim 31, wherein:
(a) the patient is administered with a dose of the plasminogen in the range of 0.01 to 100 mg/kg body weight during the treatment period; and subsequently
(b) (i) the level of plasminogen in the patient's blood is determined and,
(ii) the patient is administered with a sufficient amount of plasminogen to prevent or treat a thrombotic event if the determined level of plasminogen is at least 10% (mol/mol) lower in comparison to the average level found throughout a population of the same species.

59. The method of claim 31, wherein the patient suffers from deep vein thrombosis, pelvic vein thrombosis, pulmonary embolism, an infarction of any organ, retinal vein occlusion, disseminated intravascular coagulation (DIC), thrombotic thrombocytopenic purpura (TTP), an angiopathy coincidence with thrombotic events in capillary flow path, diabetic angiopathy, thrombophlebitis, or a combination of two or more thereof.

60. The method of claim 31, wherein the patient is at risk of developing a thrombotic event due to atherosclerosis or stenosis of arteries, having been subjected to a surgery, or having an implanted blood vessel endoprosthesis.

61. The method of claim 31, wherein the patient suffers from disseminated intravascular coagulation (DIC), acute kidney/renal injury (AKI), sepsis, or a combination of two or more thereof.

62. The method of claim 58, wherein steps (i) and (ii) are conducted repeatedly as long as the level of plasminogen determined in step (i) is at least 10% (mol/mol) lower in comparison to the average level found throughout the population of the same species.
